(19) United States
(12) Patent Application Publication
(10) Pub. No.: US 2022/0219993 A1
(43) Pub. Date: Jul. 14, 2022

(54) POROUS NANOSHEETS FOR EFFECTIVE ADSORPTION OF SMALL MOLECULES AND VOLATILE ORGANIC COMPOUNDS

(71) Applicant: Nanyang Technological University, Singapore (SG)

(72) Inventors: Jing-Hao CIOU, Singapore (SG); Pooi See LEE, Singapore (SG)

(21) Appl. No.: 17/607,753

(22) PCT Filed: May 29, 2020
(86) PCT No.: PCT/SG2020/050317

§ 371 Date: Oct. 29, 2021

(30) Foreign Application Priority Data
May 29, 2019 (SG) 10201904893T

(51) Int. Cl. C01B 32/921; B01J 20/02; B01J 20/28; B01J 20/282; B01D 53/04; G01N 33/00; G01N 5/02; G01N 21/31; H05B 3/12

(52) U.S. Cl. CPC C01B 32/921; H05B 2203/013; B01J 20/28035; B01J 20/282; B01D 53/0438; G01N 33/0047; G01N 5/02; G01N 21/31; H05B 3/12; B01J 2220/42; C01P 2004/04; C01P 2002/72; C01P 2002/82; C01P 2004/03; C01P 2002/85; B01D 2257/708; B01D 2253/10; B01D 2253/304; B01D 2259/40096; B01J 20/0211

(57) ABSTRACT

Disclosed herein is a material suitable for the adsorption, storage and release of volatile organic compounds comprising: a porous thin film layer formed from nanosheets of one or more MXenes.

### FIELD OF INVENTION

The current invention relates to a material suitable for the adsorption, storage and release of volatile organic compounds (VOCs). It also relates to a volatile organic compound storage device comprising said material.

### BACKGROUND

The listing or discussion of a prior-published document in this specification should not necessarily be taken as an acknowledgement that the document is part of the state of the art or is common general knowledge.

Detection of volatile organic compounds (VOCs) is increasingly important in applications such as monitoring of plant health. It is essential to monitor and detect pathogens to minimise the spread of plant diseases induced by pests, fungi, bacteria or viruses. Existing methods to monitor pathogen infection of plants include serological assays such as enzyme-linked immunosorbent assays (ELISA) and western blots. These methods detect phytopathogens by antibody-antigen interactions or the formation of protein-antibody-antibody complexes. There are also nucleic acid-based methods, such as fluorescence in-situ hybridization and polymerase chain reaction (PCR), which can detect targeted DNA sequence by pre-designed primers. However, as these methods directly detect pathogens, they are only useful when plants are symptomatic.

Indirect detection methods are now of interest to monitor early-stage plant infections. Relying on the metabolites of pathogens as biomarkers, infections can be detected at the asymptomatic stage. The methods to detect metabolites including gas chromatography-mass spectrometry (GCMS) (B. Warth et al., Metabolomics 2015, 11 (3), 722-738; D. Gomathi et al., Journal of food science and technology 2015, 52 (2), 1212-1217), surface-enhanced Raman scattering spectroscopy (SERS) (N. N. Durmanov et al., Sensors and Actuators B: Chemical 2018, 257, 37-47), electronic nose (E-nose) (A. Cellini et al., Sensors 2017, 17 (11), 2596), and lateral flow microarrays (LFM) (D. J. Carter, R. B. Cary, Nucleic acids research 2007, 35 (10), e74). These techniques can identify VOC profiles or proteins, which reveal “plant-to-pathogen” or “plant-to-plant” interactions. While GCMS, SERS and LFM can provide detection with shortened time as compared to PCR, they cannot provide in-situ monitoring (or real time detection) of plant health conditions. Similarly, while E-nose provides near-real time monitoring, it lacks reproducibility and suffers from poor resolution. There is therefore a need for improved materials and methods for detecting small molecules and/or VOCs that solves one or more problems mentioned above.

The adsorption and control release of molecules has been widely used in healthcare, agriculture, food industry as well as the human machine interaction. In these fields, the molecules carry specific information which induces corresponding specific responses, such as immune response, seed germination, the synthesis of nutrient or even arousing the deep mind emotion of human. However, these physiological responses from the organisms can be only triggered when an appropriate level of the molecule in question has accumulated. Thus, the means to keep or trap and to release the molecules in a controlled manner is crucial for achieving the specific functions. This is especially important for aroma molecules, as their volatile nature allows them to be vaporized easily at ambient temperature. So far, the controlled release of the aroma molecules is commonly applied in food science, indoor air quality improvement and olfactory display.

Many approaches and materials have been designed to provide the controllable release and preservation of aroma molecules. For example, protection of aroma molecules has been studied by a microencapsulation system consisting of metal organic frameworks, polymers, starch, gums, proteins or lipids. Aroma molecules are then released when the microencapsulation system is triggered by suitable conditions, such as temperature, pH value or the addition of kinases.

So far, the distribution of aroma molecules for olfactory display by heating, light, and mechanical force has been studied. The thermal controlled release method (or temperature-induced release) has the most potential to be combined with audial or visual technologies. The thermal controlled release method typically involves the use of an external heating source to perform the thermal release of aroma molecules. However, by using an external heating source, undesirable effects such as the time delay of the molecule release and the thermal degradation/aging of the adsorbent material (especially for an organic or a polymeric based adsorbent) are unavoidable. Thus, there remains a need for a material with improved properties that can function in an olfactory display.

### SUMMARY OF INVENTION

It has been surprisingly found that nanosheets of MXenes can act to encapsulate molecules and this material can also act as the heating element/heating source in order to improve the distribution of aroma molecules in an olfactory display.

Aspects and embodiments of the current invention will now be described in the following numbered clauses.

1. A material suitable for the adsorption, storage and release of volatile organic compounds comprising:
a porous thin film layer formed from nanosheets of one or more MXenes.

2. A volatile organic compound storage device, comprising:
a material suitable for the adsorption, storage and release of volatile organic compounds comprising a porous thin film layer formed from nanosheets of one or more MXenes; and
electrodes attached to a surface of the porous thin film layer formed from nanosheets of one or more MXenes.

3. The volatile organic compound storage device according to Clause 2, wherein:

(aa) the electrodes may be provided in the form of a metal tape on a surface of the porous thin film layer formed from nanosheets of one or more MXenes, optionally wherein the metal tape is selected from one or more of gold, silver and copper, such as copper; and/or

(bb) the volatile organic compound storage device may be configured to release a volatile organic compound stored in said device by way of resistive heating.

4. The material according to Clause 1 or the volatile organic compound storage device according to Clause 2 or Clause 3, wherein the material may be provided solely as a free-standing porous thin film layer.

5. The material according to Clause 1 or the volatile organic compound storage device according to Clause 2 or Clause 3, wherein the material may further comprise a substrate material having a surface and the porous thin film layer may be formed on the surface of the substrate material.

6. The volatile organic compound storage device or the material according to Clause 5, wherein the substrate material may be selected from one or more of the group consisting of silicon, glass, polyethylene terephthalate, mica, and anodic aluminium oxide.

7. The material according to Clauses 1 and Clauses 4 to 6 or the volatile organic compound storage device according to any one of Clauses 2 to 6, wherein the one or more MXenes may have a minimum lateral size of 0.22 μm, optionally wherein the one or more MXenes may have a minimum lateral size of from 0.22 μm to 1 μm.

8. The volatile organic compound storage device or the material according to Clause 7, wherein the one or more MXenes may have a minimum lateral size of 0.45 μm, such as a lateral size of from to 0.45 μm to 1 μm.

9. The material according to any one of Clause 1 and Clauses 4 to 8 or the volatile organic compound storage device according to Clauses 2 to 8, wherein each nanosheet may have a thickness of from 1 nm to 6 nm, such as from 1.5 nm to 4 nm.

10. The volatile organic compound storage device or the material according to Clause 9, wherein each nanosheet may have a thickness of about 2 nm.

11. The material according to any one of Clause 1 and Clauses 4 to 10 or the volatile organic compound storage device according to Clauses 2 to 10, wherein the lateral size of each nanosheet may be from 0.45 μm to 10 μm, such as from 0.5 μm to 5 μm.

12. The volatile organic compound storage device or the material according to Clause 11, wherein the lateral size of each nanosheet may be from 1 μm to 2 μm.

13. The material according to any one of Clause 1 and Clauses 4 to 12 or the volatile organic compound storage device according to Clauses 2 to 12, wherein the one or more MXenes may be selected from one or more of the group consisting of Ti₂C, (Ti_{0.5}, Nb_{0.5})₂C, V₂C, Nb₂C, M0₂C, Mo₂N, (Ti_{0.5}, Nb_{0.5})₂C, Ti₂N, W_{1.33}C, Nb_{1.33}C, Mo_{1.33}C, MO_{1.33}Y_{0.67}C, Ti₃C₂, Ti₃CN, Zr₃C₂, Hf₃C₂, Ti₄N₃, Nb₄C₃, Ta₄C₃, V₄C₃, Mo₄VC₄, Mo₂TiC₂, Cr₂TiC₂, Mo₂ScC₂, and Mo₂Ti₂C₃.

14. The volatile organic compound storage device or the material according to Clause 13, wherein the one or more MXenes may be selected from one or more of the group consisting of Ti₂C, Nb₄C₃, Mo₂C, and Ti₃C₂.

15. The volatile organic compound storage device or the material according to Clause 14, wherein the one or more MXenes may be Ti₃C₂.

16. The material according to any one of Clause 1 and Clauses 4 to 15 or the volatile organic compound storage device according to Clauses 2 to 15, wherein the thickness of the porous thin film layer may be from 5 μm to 20 μm, such as from 8 μm to 15 μm, such as from 9 μm to 11 μm.

17. The material according to any one of Clause 1 and Clauses 4 to 16 or the volatile organic compound storage device according to Clauses 2 to 16, wherein:

(BBA) the MXenes may be chemically modified by reaction with a hydrophobic molecule, optionally wherein the hydrophobic molecule is an organosilane (e.g. the organosilane is selected from one or more of the group consisting of (3-aminopropyl)trimethoxysilane (APTMS), trimethoxy(octyl)silane (TEOS), trimethoxy(propyl)silane (TEPS), and trimethoxymethylsilane (TEMS)); and/or

(BBB) a BET surface area of the porous thin film layer formed from nanosheets of one or more MXenes may be from 150 to 250 m²/g, such as from 180 to 200 m²/g, such as 182.32m²/g.

18. A method of forming a free-standing porous thin film layer of an MXene, comprising the steps of:

(a) providing a suspension of porous nanosheets of an MXene in a solvent;

(b) subjecting the suspension of porous nanosheets of an MXene to vacuum filtration with a filter membrane to provide a porous thin-film layer on a surface of the filter membrane; and

(c) removing the porous thin-film layer from the surface of the filter membrane to provide the free-standing porous thin film layer of an MXene.

19. The method according to Clause 18, wherein:

(ai) the filter membrane may be a polyvinylidene fluoride membrane or a polycarbonate membrane; and/or

(aii) the filter membrane may be porous and has a pore size of from 0.22 μm to 1 μm, such as from 0.45 to 1 μm, such as from 0.22 μm to 0.45 μm; and/or

(aiii) the solvent in the suspension of porous nanosheets of an MXene may be water; and/or

(aiv) the method may further comprise a precursor step (oa), where the suspension of porous nanosheets of an MXene in a solvent is reacted with a hydrophobic molecule before steps (b) and (c) are conducted, optionally wherein the hydrophobic molecule is an organosilane (e.g. the organosilane is selected from one or more of the group consisting of (3-aminopropyl)trimethoxysilane (APTMS), trimethoxy(octyl)silane (TEOS), trimethoxy(propyl)silane (TEPS), and trimethoxymethylsilane (TEMS)).

20. A method of forming a porous thin film layer of an MXene on a surface of a substrate material, the method comprising the steps of:

(A) providing a suspension of porous nanosheets of an MXene in a first solvent;

(B) adding the suspension to a second solvent, where the first and second solvents are immiscible, to provide the porous nanosheets of an MXene at an interface between the first and second solvents;

(C) collecting the porous nanosheets of an MXene from the interface of the first and second solvents using a surface of a substrate material to which the porous nanosheets of an MXene are attachable to, to provide the porous thin film layer of an MXene on a surface of a substrate material.

21. The method according to Clause 20, wherein:

(Ai) the second solvent and suspension of porous nanosheets of an MXene may be used in a Volume:Volume ratio of from 250:1 to 500:1 (e.g. 500:1);

(Aii) the first solvent may be water and the second solvent may be chloroform; and/or

(Aiii) a third solvent that is miscible with the first and second solvents may form part of the suspension of porous nanosheets of an MXene in a first solvent, optionally wherein:
(AA) the third solvent may be an organic alcohol, such as methanol or ethanol (e.g. methanol); and/or
(BB) the Volume:Volume ratio of the first solvent to third solvent may be 1:1; and/or
(CC) the Volume:Volume ratio of the third solvent to second solvent may be 1:500; and/or
(DD) the Volume:Volume ratio of the combined first and third solvents to the second solvent may be 1:250; and/or

(Aiv) the substrate material may be formed from one or more of the group consisting of silicon, glass, polyethylene terephthalate, mica, and anodic aluminium oxide; and/or

(Av) the method may further comprise a precursor step (OA), where the suspension of porous nanosheets of an MXene in a solvent is reacted with a hydrophobic molecule before steps (B) and (C) are conducted, optionally wherein the hydrophobic molecule is an organosilane (e.g. the organosilane is selected from one or more of the group consisting of (3-aminopropyl)trimethoxysilane (APTMS), tri methoxy(octyl)si lane (TEOS), trimethoxy(propyl)silane (TEPS), and trimethoxymethylsilane (TEMS)).

22. The method of forming a free-standing porous thin film layer of an MXene according to Clause 18 or Clause 19 or the method of forming a porous thin film layer of an MXene on a surface of a substrate material according to Clause 20 or Clause 21, wherein the MXene in the free-standing porous thin film layer of an MXene or the porous thin film layer of an MXene on a surface of a substrate material:

(i) may have a minimum lateral size of 0.22 μm, optionally wherein MXene has a minimum lateral size of from 0.22 μm to 1 μm, such as a minimum lateral size of 0.45 μm, such as a lateral size of from to 0.45 μm to 1 μm; and/or

(ii) may be provided in the form of nanosheets and each nanosheet has a thickness of from 1 nm to 6 nm, such as from 1.5 nm to 4 nm, such as a thickness of about 2 nm; and/or

(iii) may be provided in the form of nanosheets, where the lateral size of each nanosheet is from 0.45 μm to 10 μm, such as from 0.5 μm to 5 μm, such as from 1 μm to 2 μm; and/or

(iv) may be selected from one or more of the group consisting of Ti₂C, (Ti_{0.5}, Nb_{0.5})₂C, V₂C, Nb₂C, Mo₂C, Mo₂N, (Ti_{0.5}, Nb_{0.5})₂C, Ti₂N, W_{1.33}C, Nb_{1.33}C, Mo_{1.33}C, Mo_{1.33}Y_{0.67}C, Ti₃C₂, Ti₃CN, Zr₃C₂, Hf₃C₂, Ti₄N₃, Nb₄C₃, Ta₄C₃, V₄C₃, Mo₄VC₄, Mo₂TiC₂, Cr₂TiC₂, Mo₂ScC₂, and Mo₂Ti₂C₃ (e.g. the MXene is selected from one or more of the group consisting of Ti₂C, Nb₄C₃, Mo₂C, and Ti₃C₂, optionally wherein the MXene is Ti₃C₂; and/or

(v) the thickness of the porous thin film layer may be from 5 μm to 20 μm, such as from 8 μm to 15 μm, such as from 9 μm to 11 μm.

23. A method of forming a volatile organic compound storage device, comprising the step of forming two or more electrodes on a surface of:

(AA) a freestanding porous thin film layer formed from nanosheets of one or more MXene; or

(AB) a porous thin film layer formed from nanosheets of one or more MXene on a substrate.

24. The method of forming a volatile organic compound storage device according to Clause 23, wherein the one or more Mxenes in the free-standing porous thin film layer of one or more MXenes or the porous thin film layer of one or more MXenes on a surface of a substrate material:

(iA) may have a minimum lateral size of 0.22 μm, optionally wherein the one or more MXenes has a minimum lateral size of from 0.22 μm to 1 μm, such as a minimum lateral size of 0.45 μm, such as a lateral size of from to 0.45 μm to 1 μm; and/or

(iiA) may be provided in the form of nanosheets and each nanosheet has a thickness of from 1 nm to 6 nm, such as from 1.5 nm to 4 nm, such as a thickness of about 2 nm; and/or

(iiiA) may be provided in the form of nanosheets, where the lateral size of each nanosheet is from 0.45 μm to 10 μm, such as from 0.5 μm to 5 μm, such as from 1 μm to 2 μm; and/or

(ivA) may be selected from one or more of the group consisting of Ti₂C, (Ti_{0.5}, Nb_{0.5})₂C, V₂C, Nb₂C, Mo₂C, Mo₂N, (Ti_{0.5}, Nb_{0.5})₂C, Ti₂N, W_{1.33}C, Nb_{1.33}C, Mo_{1.33}C, Mo_{1.33}Y_{0.67}C, Ti₃C₂, Ti₃CN, Zr₃C₂, Hf₃C₂, Ti₄N₃, Nb₄C₃, Ta₄C₃, V₄C₃, Mo₄VC₄, Mo₂TiC₂, Cr₂TiC₂, Mo₂ScC₂, and Mo₂Ti₂C₃ (e.g. the one or more MXenes is selected from one or more of the group consisting of Ti₂C, Nb₄C₃Mo₂C, and Ti₃C₂, optionally wherein the one or more MXenes is Ti₃C₂; and/or

(VA) the thickness of the porous thin film layer may be from 5 μm to 20 μm, such as from 8 pm to 15 μm, such as from 9 μm to 11 μm.

25. A method of detecting an analyte comprising the steps of:

(BA) exposing a material suitable for the adsorption, storage and release of volatile organic compounds as described in any one of Clause 1 and Clauses 4 to 17 to an environment where the analyte is suspected to be present for a period of time; and

(BB) subsequently subjecting the material suitable for the adsorption, storage and release of volatile organic compounds to spectroscopic and/or gravimetric analysis to determine the presence or absence of the analyte in said environment.

26. The method according to Clause 25, wherein the spectroscopic analysis method may be Raman spectroscopy and/or FT-IR spectroscopy.

27. The method according to Clause 25 or Clause 26, wherein the analyte may be a volatile organic compound, optionally wherein the volatile organic compound may be selected from one or more of α-pinene, 1-hexanol, a terpinol, and phenethyl alcohol.

28. The method according to any one of Clauses 25 to 27, wherein the material suitable for the adsorption, storage and release of volatile organic compounds may be in the form of a volatile organic compound storage device as described in any one of Clauses 2 to 17, and the method further comprises removing the analyte from the material suitable for the adsorption, storage and release of volatile organic compounds by application of resistive heating.

29. An olfactory display system comprising at least one volatile organic compound storage device as described in any one of Clauses 2 to 17.

### BRIEF DESCRIPTION OF DRAWINGS

FIG. 1 (a) TEM image of Ti₃C₂ MXene nanosheets with SAED pattern inset (the scale bar in SAED pattern is 5 nm⁻¹). (b) AFM image of double layer Ti₃C₂ MXene nanosheets deposited on the silicon wafer by spin coating. The thickness profile is attached and showed the height difference corresponding to the white line. (c) XRD pattern of the precursor Ti₃AlC₂ and Ti₃C₂ MXene paper.

FIG. 2 (a) In situ XRD pattern is presented by stacking multiple scans of Ti₃C₂ MXene during the adsorption of phenethyl alcohol (PA). Below the XRD pattern, a color map corresponding to the peak intensity of (002) peak shows the significant peak shift downward to lower 2 theta region during the adsorption of PA. (b) and (c) XPS spectra of the Ti₃C₂ MXene paper before (b) and after (c) adsorption of PA at O 1s core level. (d) FTIR spectra of pristine Ti₃C₃ MXene paper, PA/Ti₃C₂ paper and pure PA compound. (e) Amount of PA uptake by pristine Ti₃C₂ MXene paper from 25° C. to 100° C.

FIG. 3 Raman spectra of Si substrate, Ti₃C₂MXene before and after 1-hexanol adsorption, and 1-hexanol confirming effective adsorption of 1-hexanol on Ti₃C₂ MXene for enhanced Raman signal on silicon substrate (after 12 hours of molecular adsorption/trapping). The bare Si without MXene does not give Raman signal for the same 1-hexanol exposure.

FIG. 4 FTIR spectra confirming surface modifications of Ti₃C₂MXene porous nanosheets using 3-aminopropyl)trimethoxysilane (APTMS), trimethoxy(octyl)silane (TEOS), trimethoxy(propyl)silane (TEPS) and trimethoxymethylsilane (TEMS).

FIG. 5 FTIR spectra confirming α-Pinene adsorption by TEPS-modified Ti₃C₂ MXene (TEPS_MX). FTIR spectra of pristine Ti₃C₂ MXene (p_MX), TEPS-modified Ti₃C₂ MXene before (TEPS_MX) and after adsorption of alpha-pinene (TEPS_MX-Pin) are also provided.

FIG. 6 XRD patterns of graphene paper before and after PA adsorption.

FIG. 7 N₂ adsorption and desorption isotherms of Ti₃C₂MXene paper and graphene paper. The type IV isotherm and H2a hysteresis loop indicate the monolayer and multilayer adsorption, accompanying with capillary condensation in Ti3C2 MXene paper. In the submonolayer coverage state of adsorbed gas, the pore width can be expanded due to the decrease of solid-fluid potential between the adsorbate molecules in the sub-monolayer. The phenethyl molecules (PA) may first adsorbed on the larger mesopores followed by the micropore expansion and then further intercalated into the layer space. In addition, the expanded interlayer space was calculated as ≈5.93 Å, which may be close to the kinetic diameter of phenethyl alcohol molecules.

FIG. 8 (a) XRD pattern showing the (002) peak position of PA/Ti₃C₂ paper over a 4-month period compared with pristine Ti₃C₂ paper. (b) The d-spacings calculated from each peak position based on Bragg's law. (c) The wider 2 theta range of XRD pattern shows the good air stability of PA/Ti₃C₂ and pristine Ti₃C₂ paper.

FIG. 9 (a) IR images show the resistive heating performance of the Ti₃C₂ MXene paper from 1.0 V to 2.5 V. (b) The temperature profile presents the heating stability of Ti₃C₂ MXene paper over a period of 20 minutes.

FIG. 10 (a) IR images show the resistive heating performance of graphene paper from 1.0 V to 2.5 V. (b) The temperature profile presents the heating stability of Ti₃C₂ MXene paper over a period of 20 minutes.

FIG. 11 (a) XRD pattern of PA/Ti₃C₂ paper applied with different voltage levels for one minute. (b) The d-spacings calculated from each peak position based on Bragg's law.

FIG. 12 SEM images of Ti₃C₂ MXene nanosheets deposited on (a) Si wafer and on (b) gold nanoparticle (the dash line depicts an example of a single nanosheet of Ti₃C₂ MXene, and there are many nanosheets on the surface as reflected by the contrast shown).

FIG. 13 In-situ XRD pattern (left) and color map (right) of PA/Ti₃C₂ MXene paper confirming stable molecule adsorption in ambient conditions over 5 hours.

FIG. 14 BET N₂ adsorption and desorption isotherm of (a) Ti₃AlC₂, (b) Ti₃C₂ MXene powder, and (c) Ti₃C₂ MXene paper. The pore size distribution of Ti₃C₂ MXene and Ti₃AlC₂ are also provided; (d) Ti₃AlC, (e) Ti₃C₂-Powder and (f) Ti₃C₂-Paper.

FIG. 15 Wide range XPS spectrum of TEPS modified Ti₃C₂ MXene.

FIG. 16 Wide range XPS spectrum of pristine Ti₃C₂ MXene.

FIG. 17 Schematic of Ti₃C₂ MXene paper having electrodes at opposing ends for use as a heater.

FIG. 18 Schematic for the formation of Ti₃C₂ MXene paper from precursor, and use of the material for the adsorption and release of phenthyl alcohol (PA).

### DESCRIPTION

It has been surprisingly found that MXene materials can act to releasably store volatile organic compounds, which makes them useful in a range of different application, which include, but are not limited to olfactory display systems.

Thus, in a first aspect of the invention, there is provided a volatile organic compound storage device, comprising:
a material suitable for the adsorption, storage and release of volatile organic compounds comprising a porous thin film layer formed from nanosheets of one or more MXenes; and
electrodes attached to a surface of the porous thin film layer formed from nanosheets of one or more MXenes.

As noted above, the device may be suitable for use in an olfactory display system, amongst other uses. In essence, the MXene provides a dual function in the device. Its first role is to provide adsorption sites for the capture of volatile organic compounds (e.g. aroma molecules). The MXenes can then also act as the heating source that enables the controlled release of captured volatile organic compounds (e.g. aroma molecules). Due to abundant termination groups on the surface and its metallic nature, MXenes provide a tremendous number of active sites for interaction with and capture of volatile organic compounds. Additionally, MXene can be electrically heated to thermally desorb the aroma molecules from the interaction sites. This approach eliminates the interface incompatibility issues that currently exist between the heating source and the molecular encapsulation layer in conventional olfactory display system.

Some advantages associated with the device described hereinbefore, which are also associated with the nanosheets of one or more MXenes, include:

1. the porous nanosheet holds long-term stability for molecular storage;

2. the recovery or release of adsorbed molecules can be done by resistive heating, whereby the porous nanosheets itself act as a highly effective resistive heater; and 3. porous nanosheets can be a coating on different substrates for diagnostic detection of molecules.

These advantages are demonstrated herein by reference to a Ti₃C₂ MXene device, as discussed below.

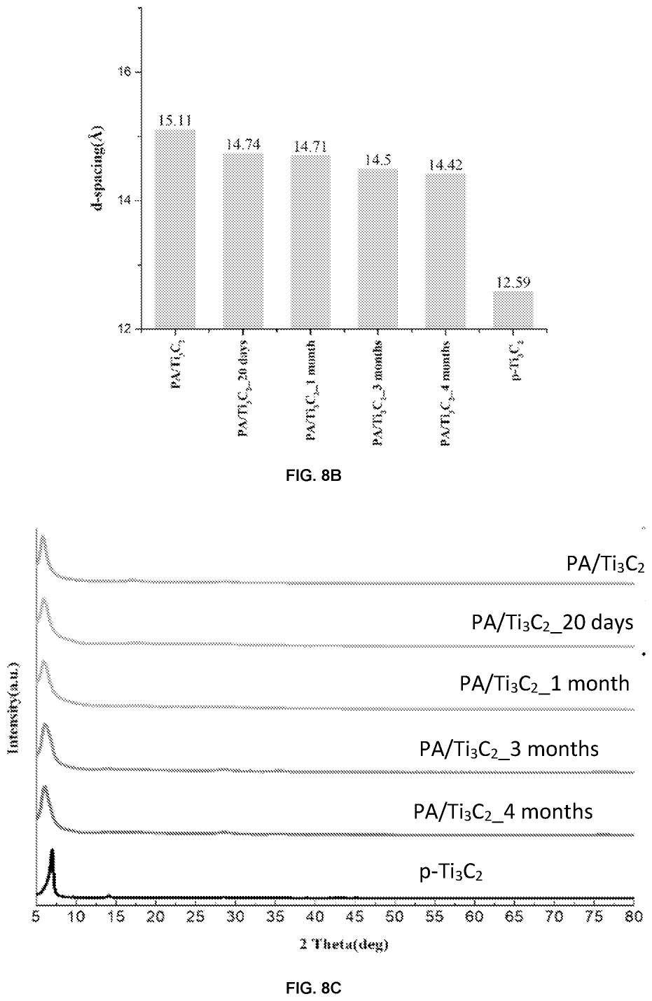

Statement 1 can be verified by the XRD pattern in the FIG. 8a. The intercalation of PA molecule in Ti₃C₂ MXene induces the expansion of interlayer spacing, which can be observed by the XRD. In FIG. 8b, the interlayer spacing of pristine Ti₃C₂ MXene was calculated to be 12.68 Å and 12.59 Å (due to experimental variation between batches). by Bragg's law. After the intercalation of PA, the interlayer spacing of Ti₃C₂ MXene increases to 15.11 Å, which, furthermore, can remain the distance at 14.42 Å over the period of 4 months. As compared to the best works till date on molecular storage in Table 1, Ti₃C₂ MXene paper is a promising and highly competitive candidate for the application in molecule storage.

TABLE 1
The comparison of the long-term storage of small molecule in different materials.

| | Chemical | Method | | Release | |
|---|---|---|---|---|---|
| | intend to | to | Stable | of | |
| Materials | store | Store | Time | molecule | Reference |
| Ti₃C₂ | Phenethyl | Interlayer | 4 months | Joule | This work |
| MXene | alcohol | intercalation | | Heating | |
| paper | | | | | |
| Paraffin | 2-acetyl-1- | Microencapsulation | 3 months | Heating | Y. Yin, K. R. |
| | pyrroline | | | | Cadwallader, |
| | | | | | Food chemistry |
| | | | | | 278, 738-743 |
| | | | | | (2019) |
| Polyurethane | benzyl | Adsorption in | 1.5 months | Evaporation | X. Chen et al., |
| Acrylate | acetate | polymer matrix | | | Flavour and |
| (PUA) | | | | | fragrance |
| | | | | | journal 34, |
| | | | | | 124-132 (2019) |
| Sorbitan | menthol | Dissolve in | 0.5 month | Heating | M. Wei et al., |
| monostearate | | polymer matrix | | | Applied |
| Guargum | | | | | Sciences 10, |
| Composite | | | | | 1677 (2020) |

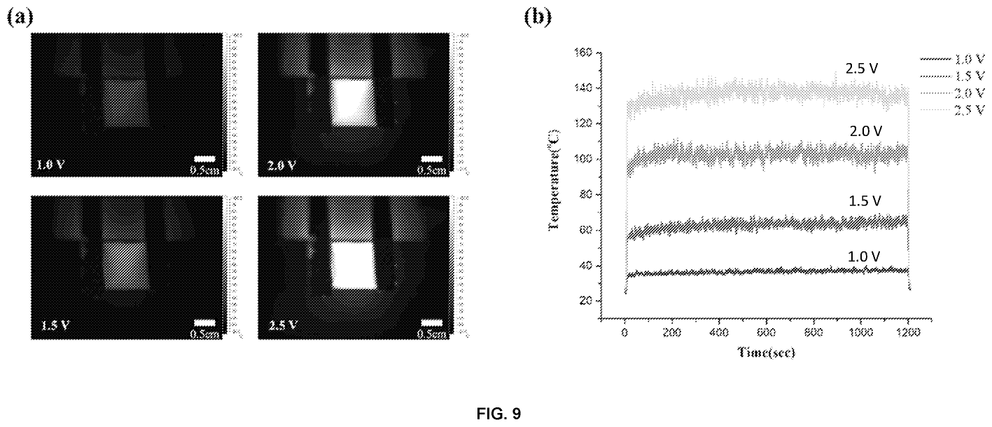

Statement 2 can be verified by the IR spectra, temperature profile and XRD pattern in the FIG. 9 and FIG. 11. The metallic nature of Ti₃C₂ MXene paper allows itself to act as a heater to perform the real time release of the encapsulated molecules upon voltage trigger. From the IR image and temperature profile in FIG. 9, Ti₃C₂ MXene paper can be heated up to several different, defined temperature levels by use of different voltages—with the desired temperature level being obtained within 1 second of the voltage being applied. The recovery of the interlayer spacing of the Ti₃C₂ MXene paper was monitored by XRD after heating at various level of voltages. The ability to perform in-situ heating and recovery of interlayer spacing allows Ti₃C₂ MXene paper to be a promising material for applications in olfactory display.

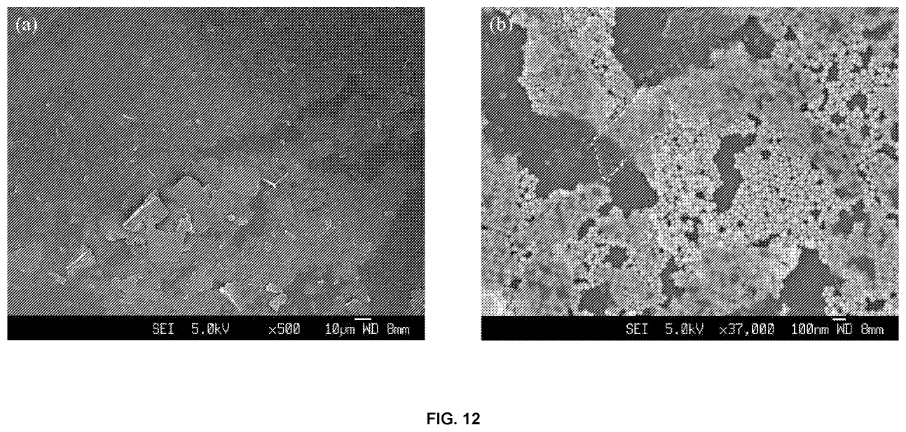

Statement 3 can be shown by the SEM images in FIG. 12. The hydrophilic nature of Ti₃C₂ MXene allows superb affinity to the hydrophilic substrate such as O₂ plasma treated Si wafer (FIG. 12a) or gold nanoparticles (FIG. 12b).

When used herein, the term “volatile organic compound” (VOC) refers to organic chemicals that have a high vapor pressure at ordinary room temperature (i.e. under ambient conditions). VOCs are numerous, varied, and ubiquitous and most scents or odors are of VOCs. Examples of VOCs include, but are not limited to esters, terpenes, organic solvents (e.g. aliphatic hydrocarbons, ethyl acetate, glycol ethers, methyl tert-butyl ether, acetone), aromatic compounds, amines, alcohols, aldehydes, ketones, lactones, thiols, chlorofluorocarbons, chlorocarbons (e.g. tetrachloroethene, dichloromethane, perchloroethylene), and the like.

Examples of esters that are VOCs include, but are not limited to, geranyl acetate, methyl formate, methyl acetate, methyl propionate, methyl propanoate, methyl butyrate, methyl butanoate, ethyl acetate, ethyl butyrate, isoamyl acetate, pentyl butyrate, pentyl butanoate, pentyl pentanoate, octyl acetate, benzyl acetate, methyl anthranilate, hexyl acetate, fructone, ethyl methylphenylglycidate, and a-methylbenzyl acetate.

Examples of terpenes that are VOCs include, but are not limited to linear terpenes (e.g. myrcene, geraniol, nerol, citral, lemonal, geranial, neral, citronellal, citronellol, linalool, nerolidol, and ocimene) and cyclic terpenes (e.g. limonene, camphor, menthol, carvone, terpineol, a-ionone, thujone, eucalyptol, and jasmone).

Examples of aromatic compounds that are VOCs include, but are not limited to benzene, benzaldehyde, eugenol, cinnamaldehyde, ethyl maltol, vanillin, anisole, anethole, estragole, thymol, 2,4,6-trichloroanisole, and substituted pyrazines.

Examples of amines that are VOCs include, but are not limited to trimethylamine, ammonia, putrescine, diaminobutane, cadaverine, pyridine, indole, and skatole. Examples of alcohols that are VOCs include, but are not limited to methanol, ethanol, propanol, furaneol, 1-hexanol, cis-3-hexen-1-ol, and menthol. Examples of thiols that are VOCs include, but are not limited to thioacetone (2-propanethione), 2-propenethiol, (methylthio)methanethiol, ethanethiol, 2-methyl-2-propanethiol, butane-1-thiol, grapefruit mercaptan, methanethiol, furan-2-ylmethanethiol, and benzyl mercaptan.

Examples of aldehydes that are VOCs include, but are not limited to acetaldehyde, hexanal, cis-3-hexenal, furfural, hexyl cinnamaldehyde, isovaleraldehyde, and anisic aldehyde, cuminaldehyde (4-propan-2-ylbenzaldehyde). Examples of ketones that are VOCs include, but are not limited to acetone, cyclopentadecanone, dihydrojasmone, oct-1-en-3-one, 2-acetyl-1-pyrroline, and 6-acetyl-2,3,4,5-tetrahydropyridine. Examples of lactones that are VOCs include, but are not limited to gamma-decalactone, gamma-nonalactone, delta-octalactone, jasmine lactone, massoia lactone, wine lactone, and sotolon.

Examples of other compounds that are VOCs include, but are not limited to methylphosphine, dimethylphosphine, phosphine, diacetyl, acetoin, nerolin, and tetrahydrothiophene.

Specific VOCs that may be mentioned herein include, but are not limited to α-pinene, 1-hexanol, a terpinol, and phenethyl alcohol.

As will be appreciated certain VOCs may fit into more than one category above.

Any suitable material may be used as an electrode on the surface of the porous thin film layer formed from nanosheets of one or more MXenes. A suitable material that may be mentioned herein is a metal tape, such that the electrodes may be provided in the form of a metal tape on a surface of the porous thin film layer formed from nanosheets of one or more MXenes. Any suitable metal may be used as the metal tape. For example, the metal tape may be selected from one or more of gold, silver and copper. In particular embodiments that may be mentioned herein, the metal tape may be copper.

As will be appreciated, the volatile organic compound storage device disclosed herein may be configured to release a volatile organic compound stored in said device by way of resistive heating. This may be accomplished by passing an electrical current through the electrodes on the surface of the porous thin film layer formed from nanosheets of one or more MXenes. This methodology will be discussed in more detail in the experimental section below.

An olfactory display is a device that generates scents using a specific components and concentrations and provides it to the human olfactory organ, so that a desired smell is produced and detected. In combination with an odour sensing system, an olfactory display becomes a part of system that records and reproduces odours. Literature describing current progress in olfactory display systems include: D. W. Kim, et al., ICECS 2009. 16th IEEE International Conference on, IEEE: 2009; pp 703-706 and J. Amores, et al., 2018 40th Annual International Conference of the IEEE Engineering in Medicine and Biology Society (EMBC), IEEE: 2018; pp 5131-5134.

Typically, an olfactory display system comprises an odorous gas generator, a gas blender, a gas releaser along with an embedded control component. The volatile organic compound storage device disclosed herein may be integrated in the olfactory display system in various ways. For example, the volatile organic compound storage device may be integrated into a gas releaser component and controlled by the application of voltage. An example of an olfactory display system is provided by U.S. Pat. No. 9,999,698, which is incorporated herein by reference in its entirety. In particular, the volatile organic compound storage device disclosed herein functions as the fragrance source 30 referred to in U.S. Pat. No. 9,999,698. Thus, the volatile organic compound storage device disclosed herein can be substituted for the fragrance source 30 in said US patent. As such, the fully described device of U.S. Pat. No. 9,999,698 is incorporated herein with the (optionally partial) exception of the fragrance source units 30 which can be replace (in whole or in part) by the volatile organic compound storage device disclosed herein.

As will be appreciated, a number of the advantages discussed herein relate to the use of a porous thin film layer formed from nanosheets of one or more MXenes, which may also be referred to herein as an “MXene paper”. Thus, there is also disclosed a material suitable for the adsorption, storage and release of volatile organic compounds comprising:
a porous thin film layer formed from nanosheets of one or more MXenes.

The advantages discussed above for the device also apply to the porous thin film layer formed from nanosheets of one or more MXenes discussed herein. Additional advantages that may apply to the nanosheets (and to the device) include:

4. the porous nanosheets have strong preferred crystal orientation with large layered structures, large interlayer spacings, active surface functional groups and high porosity; and

5. the porous nanosheets can be functionalized to enhance or specifically trap analytes.

Again, the statements may be supported herein by reference to Ti₃C₂ MXene nanosheets.

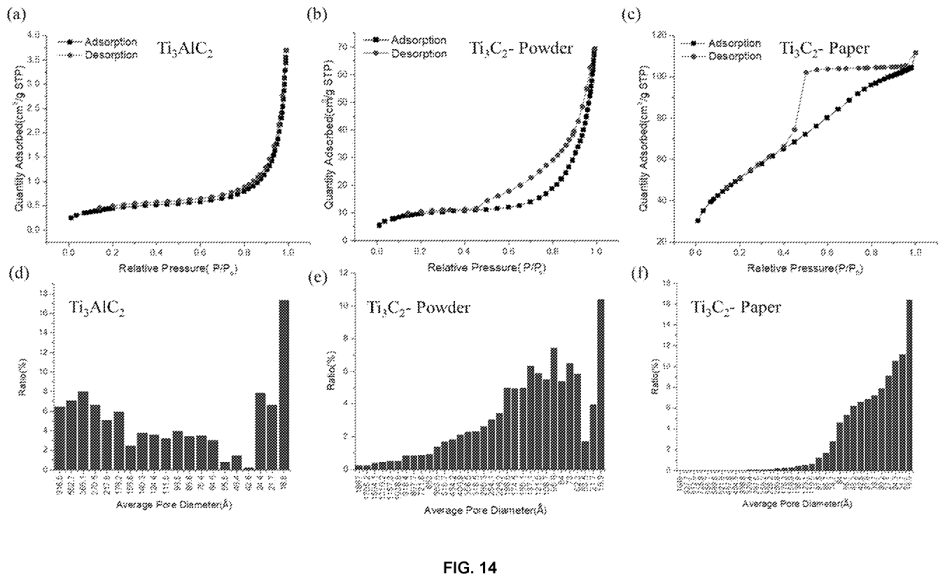

Statement 4 can be verified by the Brunner-Emmet-Teller (BET) N2 adsorption and desorption isotherm in the FIG. 14. As shown in FIG. 14b and FIG. 14c, the surface area of the Ti₃C₂ MXene is greatly improved from 32.24 m²/g to 182.32 m²/g by reassembling the nanosheets into an ordered structure. Also, from the pore size distribution histogram (FIG. 14e and FIG. 14f), the majority of the pores in the Ti₃C₂ MXene paper are sized in the mesopore region (2 nm to 50 nm), which dominates the adsorption of small molecules. The comparison of surface area with selected works that are based on two dimensional MXene material is shown below (Table 2), which supports the position that the disclosed material is a competitive porous material for the adsorption of small molecules.

TABLE 2
The comparison of surface area with selected works in form of freestanding paper.

| Materials | Surface area (m²/g) | Average pore width (Å) | Reference |
|---|---|---|---|
| Ti₃C₂ MXene paper | 182.31 | 37.9 | This work |
| Ti₃C₂Tₓ/Ag NP | 107 | N/A | L. Li et al., ACS |
| hybrid films | | | Sustainable |
| | | | Chemistry & |
| | | | Engineering 6, |
| | | | 7442-7450 (2018) |
| Ti₃C₂Tₓ | 98 | N/A | M. R. Lukatskaya |
| | | | et al., Science 341, |
| | | | 1502-1505 (2013) |

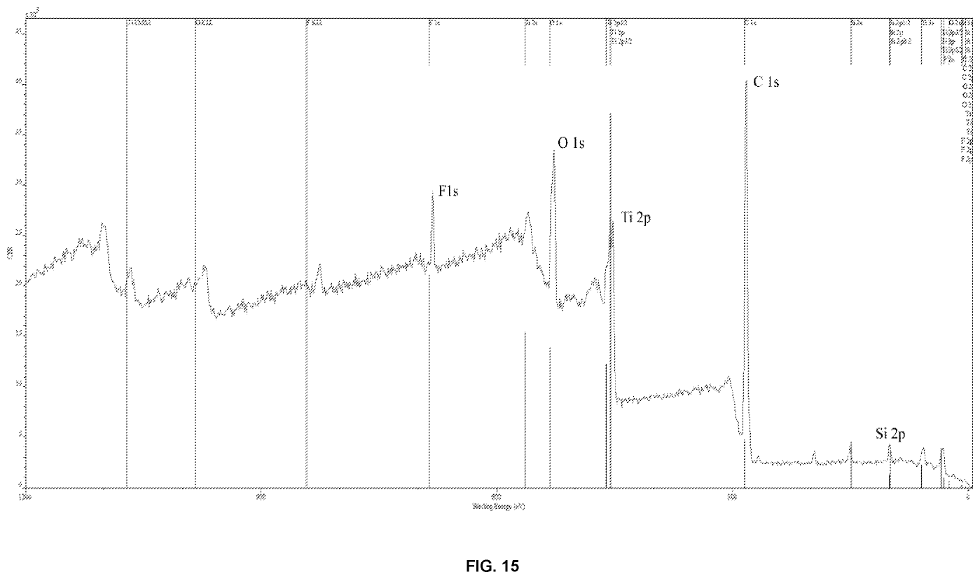

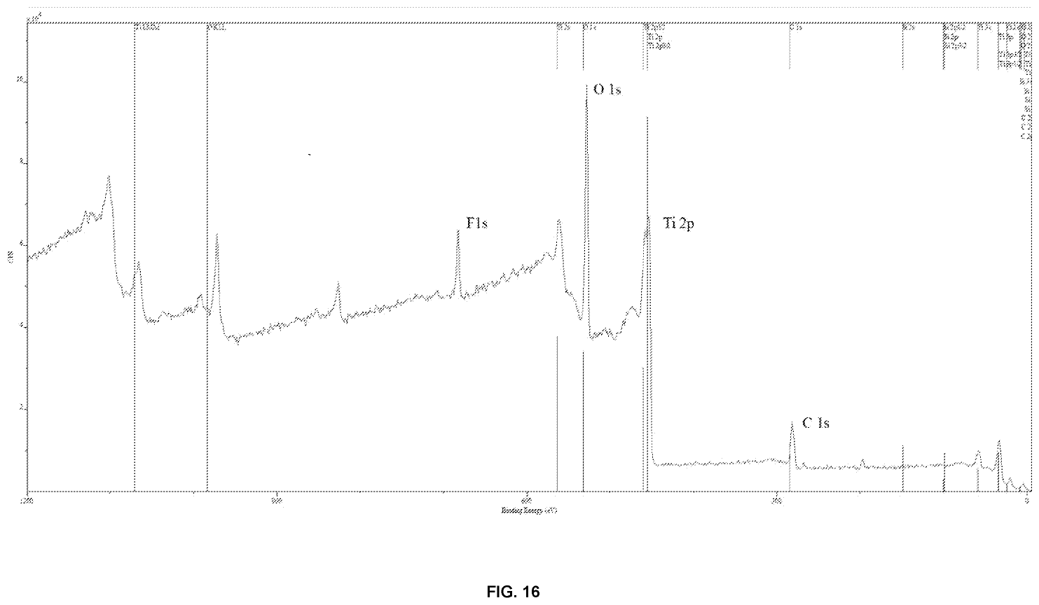

Statement 5 can be verified by the wide range XPS spectrum in FIG. 16. The abundant termination groups on the surface of Ti₃C₂ MXene provide active sites for the surface modification by various molecules. One of the example for the surface modification on Ti₃C₂ MXene is demonstrated by trimethoxypropylsilane (TEPS). The wide range XPS spectrum in FIG. 15 shows the successful surface modification with the presence of the Si 2p peak, which refers to the formation of a covalent bond between a —OH group on Ti₃C₂ MXene and the methoxy group on the TEPS.

MXenes are two dimensional materials composed of transition metal carbides, nitrides or carbonitrides, which can be generally labeled as Mₙ₊₁Xₙ (n=1 to 3) in formula. M typically represents the early transition metals such as titanium, vanadium, niobium, etc. and X typically represents carbon and/or nitrogen element. The MXene materials are obtained from the MAX ternary structure by selective etching of A layers where A usually represents the IIIA or IVA elements^{]}. After the removal of the middle A layer, the MXene surface is terminated by diverse functional groups such as —OH, —O and —F, which render the surface of MXene hydrophilic and thus provide active sites for modification and functionalization.

The MXenes used herein will be discussed in further detail below. It will be appreciated that the following discussions relating to the porous thin film layer formed from nanosheets of one or more MXenes may equally relate to said material per se or to the device comprising said material. Furthermore, it is explicitly contemplated that any technically sensible combination of the features listed below may form an embodiment of the current invention.

The porous thin film layer formed from nanosheets of one or more MXenes may be provided in two different forms, both per se and as part of the device. In the first form, the porous thin film layer formed from nanosheets of one or more MXenes may be provided solely as a free-standing porous thin film layer. In the second form, the porous thin film layer formed from nanosheets of one or more MXenes may be formed on a substrate material having a surface and the porous thin film layer may be formed on the surface of the substrate material. It will be appreciated that it is possible to form the nanosheets to contain both forms, such that a portion of the nanosheets are formed on a substrate, while a portion of nanosheets are free-standing. Additionally, a layer of the nanosheet may be used as a substrate for a further layer of said material.

When used herein, the term “free-standing” is intended to mean that the material does not need to be supported on a substrate to function and/or that it is not irreversibly attached to a substrate. For the avoidance of doubt, in the context of the current invention, electrodes, when attached to free-standing nanosheets of one or more MXenes are not to be interpreted as a substrate.

When used herein, the term “substrate” refers to a suitable surface upon which nanosheets of one or more MXenes are formed. In embodiments of the invention where the nanosheets of one or more MXenes are formed on a substrate, they may be attached to said substrate by intermolecular interactions and/or covalent bonding. Any suitable material may be used as a substrate for the nanosheets of one or more MXenes discussed herein, when said nanosheets are formed on the surface of a substrate. Suitable substrate materials include, but are not limited to silicon, glass, polyethylene terephthalate (PET), mica, and anodic aluminium oxide. Examples of other substrates that may be mentioned herein include, but are not limited to, flexible polymeric materials (which may encompass PET sheets), fabrics, filter membranes, paper, metals and the like.

When used herein, the term nanosheet may refer to a two-dimensional nanostructure with a thickness ranging from 1 to 15 nm. In more particular embodiments of the currently claimed invention, each nanosheet may have a thickness of from 1 nm to 6 nm, such as from 1.5 nm to 4 nm. For example, each nanosheet may have a thickness of about 2 nm or the average thickness of each nanosheet may be from 1 nm to 6 nm, such as from 1.5 nm to 4 nm, such as 2 nm.

The nanosheets of one or more MXenes discussed herein may have any suitable lateral size. For example, the nanosheets may have a minimum lateral size of 0.22 μm, such as from 0.22 μm to 1 μm, such as of 0.45 μm, such as a lateral size of from to 0.45 μm to 1 μm. Additionally or alternatively, the lateral size of each nanosheet may be from 0.45 μm to 10 μm, such as from 0.5 μm to 5 μm, such as from 1 μm to 2 μm.

Any suitable MXene may be used to form the nanosheets of one or more MXenes. Suitable MXenes include, but are not limited to Ti₂C, (Ti_{0.5}, Nb_{0.5})₂C, V₂C, Nb₂C, Mo₂C, Mo₂N, (Ti_{0.5}, Nb_{0.5})₂C, Ti₂N, W_{1.33}C, Nb_{1.33}C, Mo_{1.33}C, Mo_{1.33}Y_{0.67}C, Ti₃C₂, Ti₃CN, Zr₃C₂, Hf₃C₂, Ti₄N₃, p Nb₄C₃, Ta₄C₃, V₄O₃, Mo₄VC₄, Mo₂TiC₂, Cr₂TiC₂, Mo₂ScC₂, and Mo₂Ti₂C₃. It will be appreciated that a single MXene may be used to form the nanosheets. It will also be appreciated that a combination of MXenes may be used to form the nanosheets (e.g. 2, 3, 4, 5, 6, 7, 8, 9, 10 MXenes). In particular embodiments of the invention that may be discussed herein, the one or more MXenes may be selected from one or more of the group consisting of Ti₂C, Nb₄C₃, Mo₂C, and Ti₃C₂. In yet further embodiments of the invention that may be disclosed herein, the one or more MXenes may be Ti₃C₂.

As will be appreciated, the thin film layer discussed herein is formed by a plurality of the nanosheets of one or more MXenes, which may be arranged in top of one another. Thus, the thin film layer may have any suitable thickness, such as from 5 μm to 20 μm, such as from 8 pm to 15 μm, such as from 9 μm to 11 μm. For the avoidance of doubt, when multiple sets of numerical ranges are disclosed herein, the end points listed are explicitly intended to be combined with the other disclosed end-points to provide further ranges, which also forms part of the current invention. For example, the following ranges are specifically contemplated based on the multiple sets of ranges disclosed immediately above:

5 μm to 8 μm, 5 μm to 9 μm, 5 μm to 11 μm, 5 μm to 15 μm, 5 μm to 20 μm;

8 μm to 9 μm, 8 μm to 11 μm, 8 μm to 15 μm, 8 μm to 20 μm;

9 μm to 11 μm, 9 μm to 15 μm, 9 μm to 20 μm; and

15 μm to 20 μm.

The nanosheets of one or more MXenes may be chemically modified by reaction with one or more other chemical entities to provide modified chemical properties. For example, the nanosheets of one or more MXenes may be chemically modified by reaction with a hydrophobic molecule. Examples of hydrophobic molecules include, but are not limited to, an organosilane. Examples of organosilanes include, but are not limited to, (3-aminopropyl)trimethoxysilane (APTMS), trimethoxy(octyl)silane (TEOS), trimethoxy(propyl)silane (TEPS), trimethoxymethylsilane (TEMS), and combinations thereof.

The porous thin film layer formed from nanosheets of one or more MXenes may have a BET surface area of from 150 to 250 m²/g, such as from 180 to 200 m²/g, such as 182.32 m²/g.

Also disclosed herein is a method of manufacturing a free-standing porous thin film layer of an MXene. Thus, there is provided a method of forming a free-standing porous thin film layer of an MXene, comprising the steps of:

(a) providing a suspension of porous nanosheets of an MXene in a solvent;

(b) subjecting the suspension of porous nanosheets of an MXene to vacuum filtration with a filter membrane to provide a porous thin-film layer on a surface of the filter membrane; and

(c) removing the porous thin-film layer from the surface of the filter membrane to provide the free-standing porous thin film layer of an MXene.

The MXene may be suspended in any suitable solvent. An example of a suitable solvent that may be mentioned herein is water. Any suitable filter membrane may be used in step (b) above. Examples of suitable filter membranes include, but are not limited to, a polyvinylidene fluoride membrane or a polycarbonate membrane. As will be appreciated, the filter membranes may have any suitable porosity. An example of suitable pore sizes for the filter membranes are a pore size of from 0.22 μm to 1 μm, such as from 0.45 to 1 μm, such as from 0.22 μm to 0.45 μm.

In certain embodiments of the above method, there may be a further step (oa), where the suspension of porous nanosheets of an MXene in a solvent may be reacted with a hydrophobic molecule before steps (b) and (c) are conducted, optionally wherein the hydrophobic molecule is an organosilane (e.g. the organosilane is selected from one or more of the group consisting of (3-aminopropyl)trimethoxysilane (APTMS), trimethoxy(octyl)silane (TEOS), trimethoxy(propyl)silane (TEPS), and trimethoxymethylsilane (TEMS)).

Further details of the manufacture of a free-standing standing porous thin film layer of an MXene are set out in more detail in the experimental section below, which may be adapted by analogy for the various MXenes that may form part of the invention. It is noted that the all-aqueous preparation method means that there is no residual organic solvent.

As will be appreciated, the above-mentioned method is suitable for the formation of free-standing thin films, but it does not provide a porous thin film layer of an MXene on a surface of a substrate material. Thus, there is also provided a method of forming a porous thin film layer of an MXene on a surface of a substrate material, the method comprising the steps of:

(A) providing a suspension of porous nanosheets of an MXene in a first solvent;

(B) adding the suspension to a second solvent, where the first and second solvents are immiscible, to provide the porous nanosheets of an MXene at an interface between the first and second solvents;

(C) collecting the porous nanosheets of an MXene from the interface of the first and second solvents using a surface of a substrate material to which the porous nanosheets of an MXene are attachable to, to provide the porous thin film layer of an MXene on a surface of a substrate material.

When used herein, the term “attachable” means that the porous nanosheets of an MXene are bonded to the surface of the substrate. For example, the attachment may be through covalent bonding, or it may, more particularly, be through intermolecular interactions (e.g. electrostatic interactions, hydrogen bonding, Van der Waal's interactions etc.).

In the above method any two immiscible solvents may be used as the first and second solvent. As will be appreciated, it is preferable that the second solvent forms the lower portion of the immiscible solvent. Examples of a first solvent and a second solvent include water and chloroform, respectively and water and dichloromethane, respectively. The second solvent and suspension of porous nanosheets of an MXene may be used in a Volume:Volume ratio of from 250:1 to 500:1 (i.e. from 0.5:125 to 0.5:250), such as 500:1. In certain embodiments that may be mentioned herein, a third solvent that is miscible with both the first and second solvents may form part of the suspension of porous nanosheets of an MXene in a first solvent. Any suitable third solvent may be used, provided that it has some solubility in the first and second solvents. For example, if the first solvent is water and the second solvent is chloroform, the third solvent may be an organic alcohol (e.g. ethanol or, more particularly, methanol). The Volume:Volume ratio of the first solvent to third solvent may be 1:1, and the Volume:Volume ratio of the third solvent to second solvent may be 1:500 (i.e. 0.5:250). As such, the

Volume:Volume ratio of the combined first and third solvents to the second solvent may be 1:250. Without wishing to be bound by theory, the third solvent may help to accelerate the assembly of the MXene thin film.

The substrate material may be the same as discussed above in respect of the product per se. That is, the substrate material may be formed from one or more of the group consisting of silicon, glass, polyethylene terephthalate, mica, and anodic aluminium oxide.

As before, the method may further comprise a step (OA), where the suspension of porous nanosheets of an MXene in a solvent may be reacted with a hydrophobic molecule before steps (B) and (C) are conducted, optionally wherein the hydrophobic molecule is an organosilane (e.g. the organosilane is selected from one or more of the group consisting of (3-aminopropyl)trimethoxysilane (APTMS), tri methoxy(octyl)si lane (TEOS), trimethoxy(propyl)silane (TEPS), and trimethoxymethylsilane (TEMS)).

Further details of the manufacture of a porous thin film layer of an MXene on a surface of a substrate material are set out in more detail in the experimental section below, which may be adapted by analogy for the various MXenes that may form part of the invention.

Also disclosed herein is a method of forming a volatile organic compound storage device, comprising the step of forming two or more electrodes on a surface of:

(AA) a freestanding porous thin film layer formed from nanosheets of one or more MXene; or

(AB) a porous thin film layer formed from nanosheets of one or more MXene on a substrate.

As will be appreciated, the resulting porous thin film layer of an MXene described in the methods above may be derived by reference to the resulting products discussed above. Therefore, for the sake of brevity, the various embodiments of the porous thin film layer of an MXene will not be described in detail once again.

As noted above, the material disclosed herein may be useful for the capture and release of an analyte. This may find utility as part of an olfactory display or for use in the detection of an analyte. With respect to the latter, there is also disclosed a method of detecting an analyte comprising the steps of:

(BA) exposing a material suitable for the adsorption, storage and release of volatile organic compounds as described hereinbefore to an environment where the analyte is suspected to be present for a period of time; and

(BB) subsequently subjecting the material suitable for the adsorption, storage and release of volatile organic compounds to spectroscopic and/or gravimetric analysis to determine the presence or absence of the analyte in said environment.

Any suitable spectroscopic analysis method may be used. For example, the spectroscopic analysis method may be Raman spectroscopy and/or FT-IR spectroscopy.

The analyte used in the method above may be a volatile organic compound, which term has been defined hereinbefore. Examples of particular volatile organic compounds that may be mentioned herein include, but are not limited to, α-pinene, 1-hexanol, a terpinol, phenethyl alcohol, and combinations thereof.

As will be appreciated, the material suitable for the adsorption, storage and release of volatile organic compounds may be in the form of a volatile organic compound storage device as described hereinbefore, and the method further comprises removing the analyte from the material suitable for the adsorption, storage and release of volatile organic compounds by application of resistive heating.

Also disclosed herein is an olfactory display system comprising at least one volatile organic compound storage device as described hereinbefore.

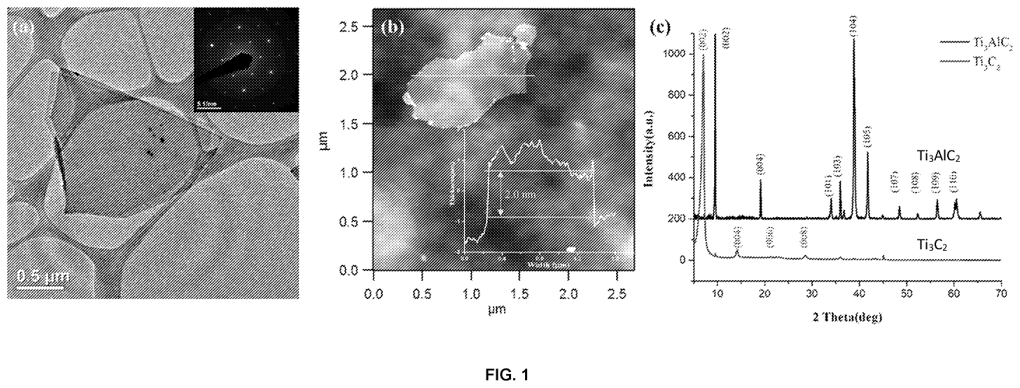

Certain aspects and embodiments of the invention are mentioned in the numbered statements below.
1. The porous nanosheets are prepared by selective etching of “A” layer of MAX phase precursor. The synthesis procedures are as follows:
   (a) 2 g of MAX phase is mixed with 40˜60 ml concentrated acidic etchant at 25° C. for 24 hr.
   (b) The mixture is repeat washed by water with centrifugation at 1000 rpm for 5 min.
   (c) The sediment is collected and stored in −20° C. to reduce the dissolved 0₂ in material.
   (d) The MXene sediment is then dispersed in 200˜400 ml DI water by bath sonication.
   (e) After sonication the suspension is subjected to centrifugation at 3000˜5000 rpm.
   (f) The supernatant is collected and bubbled with Ar gas for 5˜10 min.
   (g) The supernatant is then store in the 4° C.
2. The method in statement 1, wherein the MAX phase precursor can be Ti₃Al₂, Ti₂AlC, Nb₄AlC₃, Mo₂GaC.
3. The method in statement 1, wherein the concentrated acidic etchant can be 50% HF, 10% HF, HCl/LiF mixture.
4. The method in statement 3, wherein the HCl can be the concentration from 6˜12M.
5. After the selective etching, the resulting sediment will be frozen for 24 hours or more for temperature below water freezing point at atmospheric pressure. The MXene ice was re-dispersed and subjected to sonication to obtain the delaminated Ti₃C₂ MXene. The resulting delaminated nanosheet morphology is in layered structures as shown in FIG. 1. The TEM image of Ti₃C₂ MXene shows the bilayer Ti₃C₂ MXene with the lateral size at around 2 μm. The inset image of selected-area electron diffraction pattern demonstrates the hexagonal symmetry and the single crystallinity of Ti₃C₂ MXene (FIG. 1a). As shown by the AFM measurement, the thicknesses of Ti₃C₂ MXene nanosheets are at around 2 nm which suggest the double layer Ti₃C₂ MXene formation. The lateral size of Ti₃C₂ MXene nanosheets exhibit the compatible results with TEM at ≈1-2 μm (FIG. 1b). The XRD pattern (FIG. 1c) of the freestanding Ti₃C₂ MXene paper presents the sharp (002) peak at 2θ=7.02° which indicates the d-spacing of 12.68 Å according to the Bragg's law, and the strong [00l] orientation suggests the ordered structure constructed by the Ti₃C₂ MXene nanosheets.
6. A method to fabricate the freestanding membrane by reassembled porous nanosheets. The synthesis procedures are as follow:
   (a) Vacuum filtrate the porous nanosheet suspension with filter membrane.
7. The method in statement 6, wherein the filter membrane can be PVDF or PC membrane.
8. The method in statement 7, wherein the pore size of filter membrane can be 0.22 μm or 0.45 μm.
9. The fabrication of highly effective resistive heater utilizing the porous nanosheets: To perform resistive heating on porous nanosheet based freestanding membrane. The preparation procedures are as follow:
   (a) Use copper tape or any selected metallic component as electrode.
   (b) Adhere electrode at the two ends of freestanding membrane.
   (c) Dry in air for 24 hr.
10. The method to perform resistive heating. The procedures are as follow:
   (a) Connect both electrodes with DC power supply.
   (b) Control the voltage in the range from 1V to 3V, or other selected potential ranges.
11. The method to deposit thin film of porous nanosheet on silicon substrate. The procedures are as follow:
   (a) Use chloroform as bottom solution.
   (b) Mix the porous nanosheet suspension with methanol on 1:1 ratio.
   (c) Drop the porous nanosheet suspension on the chloroform.
   (d) Collect the reassembled porous nanosheets at the interface of two solution by silicon substrate.
   (e) Dry the substrate on hot plate at 40˜85° C.
12. The method in statement 11, wherein the solvent used to disperse the porous nanosheets must not be miscible with bottom solution.
13. The method in statement 11, wherein the solvent can be replaced by any kind of two immiscible solvent.
14. The method in statement 11, wherein the additional solvent mix with porous nanosheet suspension should be miscible with top and bottom solvent.
15. The method in statement 11, wherein the substrate to collect the reassembled porous nanosheets in the interface of two solvents can be replaced by glass slides, PET sheets, mica, anodic aluminum oxide, or other substrates etc.
16. The substrates can be decorated with nanomaterials such as nanoparticles or chemical assembly layers to enhanced the chemical or electrostatic assembly of the porous nanosheets layers.
17. The as-prepared porous nanolayers can be chemically functionalized or surface modified with the addition of chelating agents or spacers to enhance the specific binding and trapping of targeted gaseous or liquid analytes, and to increase the interlayer spacings for effective molecular trapping. For example, the chemisorption of self-assembled molecules can be carried out using (3-Aminopropyl)trimethoxysilane APTMS, Trimethoxy(octyl)silane TEOS, Trimethoxy(propyl)silane (TEPS) and Trimethoxymethylsilane (TEMS). The proven functionalization can be found using Fourier Transform Infrared Spectrometer as shown in FIG. 4.

With regard to the statements above, it is noted that they may be interpreted narrowly—that is to the specific materials mentioned therein or more broadly in keeping with the rest of the description provided herein (i.e. the statements above can be interpreted more broadly in line with the spirit and scope of the disclosure provided hereinbefore).

Additional or alternative advantages associated with the molecule storage method (and materials) disclosed herein include the following.
The porous nanosheets have strong preferred crystal orientation with large layered structures, large interlayer spacings, active surface functional groups and high porosity.
The porous nanosheet holds long-term stability of molecule storage.
The recovery or release of adsorbed molecules can be done by resistive heating, whereby the porous nanosheets itself act as a highly effective resistive heater.
Porous nanosheets can be a coating on different substrates for diagnostic detection of molecules.
Porous nanosheets can be functionalized with molecular linkers or chelating agents for enhanced or specific trapping of analytes or VOCs.

Further aspects and embodiments of the invention will now be discussed by reference to the following non-limiting examples.

### EXAMPLES

The current invention relates to the use of porous MXene nanosheets to adsorb and store potential VOCs (or potential metabolites of phytopathogens) in order to enhance detection signals. The trapped VOCs can be released by electric-induced resistive heating.

As an example, Ti₃C₂ Mxene nanosheets were produced in the form of a porous few-layer structure by a mechanical and chemical exfoliation method. The porous nanosheets can also be configured to be a thin film coating on any suitable substrate (e.g. silicon wafer, glass slides, PET sheets, fabrics, etc.).

Materials and Methods

The materials were purchased from the sources as provided below.

Ti₃AlC₂ (Famouschem Technology Co., Ltd., ≥98%)

Phenethyl alcohol (PA; Sigma-Aldrich; natural, ≥99%, FCC, FG)

LiF (Sigma-Aldrich; powder, <100 μm, 99.98% trace metal basis)

3-aminopropyl)trimethoxysilane (APTMS; Sigma-Aldrich; 97%), trimethoxy(octyl)silane (TEOS; Sigma-Aldrich; 96%), trimethoxy(propyl)silane (TEPS; Sigma-Aldrich; 97%) and trimethoxymethylsilane (TEMS; Sigma-Aldrich; 98%)

DI water (generated by Merck Milli-Q Water system with water resistivity: 18.2 MΩ·cm)

Copper tape

PVDF membrane (Durapore® PVDF). The membrane is porous and has a pore size of 0.22 μm or 0.45 μm.

FTIR spectrum was obtain by a PerkinElmer Fourier Transform Infrared spectrometer.

IR temperature was captured by a thermal imager (Fluke, Ti200).

### Example 1. Preparation of Ti₃C₂ MXene and Ti₃C₂ MXene Paper

Ti₃C₂ MXene was prepared by the selective etching of Al from Ti₃AlC₂ based on a procedure modified from M. Alhabeb et al., Chemistry of Materials 2017, 29 (18), 7633-7644.

An etchant solution was prepared by dissolving LiF (3.2 g) in 20 ml of 9 M HCl in a 60 ml plastic bottle. Ti₃AlC₂ powder (2 g) was then gradually added into the etchant solution at a molar ratio of Ti₃AlC₂:LiF=1:12 over a period of around 3 to 5 minutes. The mixture was continuously agitated by a Teflon magnetic stir bar for 24 hours at room temperature (25° C.). During the reaction, the cap of the plastic bottle was loosely screwed on the bottle to prevent buildup of pressure by the formation of hydrogen gas. The resulting sediment was washed with DI water repeatedly by 4 to 6 rounds of centrifugation at 1000 rpm for 5 minutes. The washed sediment was collected and stored in −20° C. for 24 hours to reduce dissolved O₂ in the material. The MXene sediment was then redispersed in around 200 to 400 mL of DI water by bath sonication at room temperature for 1 hour. After sonication the suspension is subjected to centrifugation at around 3000 to 5000 rpm. The supernatant was collected and bubbled with Ar gas for about 5 to 10 minutes to obtain a suspension of delaminated Ti₃C₂ MXene in few-layer structure. The supernatant was stored in −4° C.

Ti₃C₂ MXene paper (or a free-standing membrane formed from nanosheets of Ti₃C₂ MXene) was prepared by subjecting the as-obtained suspension of delaminated Ti₃C₂ MXene to vacuum filtration with a PVDF filter membrane. Typically, the PVDF membrane was placed on a funnel fixedly mounted on a filter flask connected to a vacuum pump. 30 mL of the MXene solution was poured into the funnel and with the funnel capped, the vacuum pump was then switched on and configured to reach at least around 0.8 psi of suction power. The filtration was conducted at ambient conditions. This results in around 30 mg of Ti₃C₂ MXene deposited on the membrane. The PVDF membrane was then removed to obtain free standing Ti₃C₂ MXene paper.

Transmission Electron Microscopy (TEM)

Ti₃C₂ MXene was imaged using TEM. TEM images was recorded using JEOL 2010 UHR at under 200 kV. The TEM sample was prepared by casting a drop of diluted Ti₃C₂ MXene solution on the sample holder. The sample holder was dried at vacuum desiccator at the pressure around −0.8 psi for 24 hr under room temperature. The sample holder was then mounted on the TEM sample holder and inserted into TEM for material characterization. The characterization was done under bright field mode and diffraction mode. The bright field TEM was conducted with beam current at 105 μA to 108 μA, the size of condenser aperture at 70 μm and current density around 70˜85 μA/cm². The TEM diffraction mode was conducted with camera length at 30 cm to 40 cm and the size of field limiting aperture at 20 μm or 50 μM.

As shown in FIG. 1a, the TEM image of Ti₃C₂ MXene acquired along c-axis of flakes shows a bi-layered Ti₃C₂ MXene having a lateral size of around 2 μm. The inset image of selected-area electron diffraction pattern demonstrates the hexagonal symmetry and the single crystallinity of Ti₃C₂ MXene.

Atomic Force Microscopy (AFM)

AFM (Asylum Research Cypher S) was applied to measure the thickness of Ti₃C₂ MXene. The specimen for AFM characterization was prepared by spin casting the Ti₃C₂ MXene solution on a Si wafer. The Si wafer was cleaned by acetone and isopropanol and dried with Argon gas. The dried Si wafer was then treated with O₂ plasma for 2 min with the RF power at 100 W. The spin casting of Ti₃C₂ MXene was conducted with the spin rate at 2000 rpm for 60 s. The specimen was mounted on the AFM scanning stage for the following characterization.

During the AFM scanning, the parameters were set with the scan area at 5 μm*5 μm, scan rate for 1 Hz, set point at 800mV and the integral gain to be 30. The resolution of the scanning was set to have 512 points at single line and 512 lines in total.

The thicknesses of Ti₃C₂ MXene nanosheets were measured to be around 2 nm (FIG. 1b) which suggest a double-layered Ti₃C₂ MXene based on previous studies which demonstrate that a single layer Ti₃C₂ MXene has a thickness of around ≈1 nm (X. Wang et al., Journal of the American Chemical Society 2015, 137 (7), 2715-2721; M. Ghidiu et al., Nature 2014, 516 (7529), 78). Also, the lateral size of Ti₃C₂ MXene nanosheets was in line with the TEM results at ≈1-2 μm.

X-Ray Diffraction (XRD)

XRD analysis was performed by Bruker D8 Advance to reveal the lattice plane of the material. Specifically, Ti₃C₂ MXene paper was fixed on a XRD sample holder. The sample holder was then placed on the sample bracket waiting for the sample scanning. The scan range was set from 5° to 80° . The scan rate was set to be 0.5 sec/step. The increment was set to be 0.01. The step size was set to be 0.02°.

The XRD pattern (FIG. 1c) of the freestanding Ti₃C₂ MXene paper presents the sharp (002) peak at 2θ=7.02° which indicates a d-spacing of 12.68 Å according to Bragg's law. In addition, the Ti₃C₂ MXene paper shows crystal planes along [001] without other significant peaks from different direction, which suggests an ordered structure constructed by the Ti₃C₂ MXene nanosheets. After subtraction of the d-spacing by the thickness of single layer Ti₃C₂ MXene 1 nm, the free space in between the nanosheets is estimated to be ≈2.68 Å, which is considered as the space to accommodate small molecules.

Surface Area

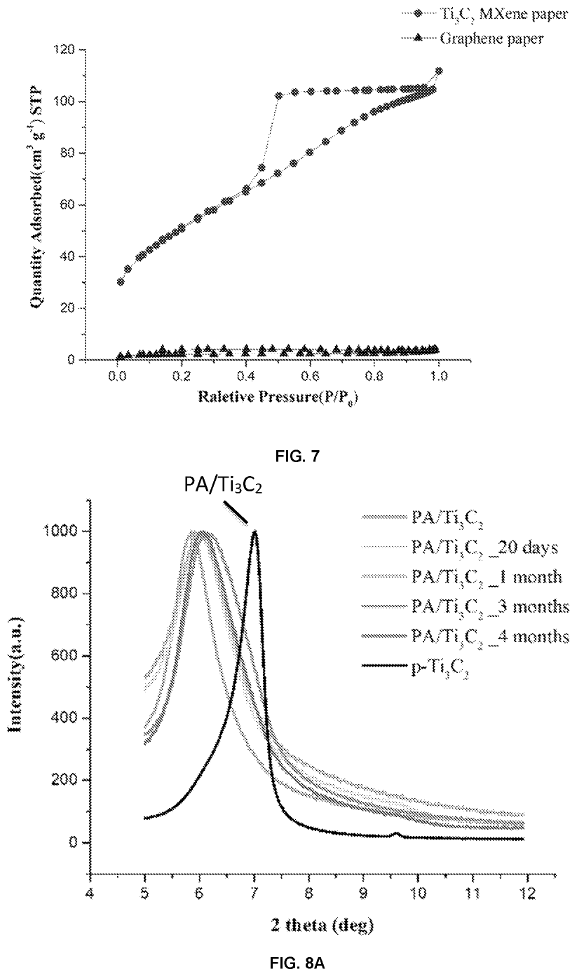

The N₂ adsorption-desorption isotherm for surface area measurement was conducted by Tristar II 3020 analyzer (FIG. 7), and the surface area was calculated by the Brunner-Emmet-Teller (BET) method. The weight of Ti₃C₂ Mxene was measured and then put into the sample tube, after which the sample tube was subjected to degas process under N₂ gas purging at 110° C. for 24 hr. Later, the sample tube was mounted on Tristar II 3020 analyzer and the sample tube was soaked into the liquid nitrogen to cool down the sample. As for the parameter setting, please refer to the user manual of Tristar II 3020 analyzer.

The calculated surface area for Ti₃C₂ MXene paper was 182.32 m²/g, which is 25 times higher than a reference material, graphene paper at 7.24 m²/g (made in accordance with procedure in Example 2).

In addition, the hysteresis loop from 0.4 to 1 p/p₀ indicates the mesoporous structure of Ti₃C₂ MXene paper. The steep desorption branch around 0.5 p/p₀ may be due to pore blocking and percolation during the evaporation of adsorbed N₂.

### Example 2. Use of Ti₃C₂ MXene Paper for Adsorption and Storage of Phenethyl Alcohol (PA)

Ti₃C₂ MXene paper was exposed to PA, an aroma compound which emits a floral odor. The interactions between PA and Ti₃C₂ MXene paper were characterised by in-situ X-ray diffraction (XRD), X-ray photoelectron spectroscopy (XPS) and Fourier-transform infrared spectroscopy (FTIR). Graphene paper was used as a reference material.

Experimental Procedure

Ti₃C₂ MXene paper (4.7 mm in diameter; prepared in accordance with Example 1) was placed and sealed in a container containing a solution of pure PA for 24 hours at room temperature. The paper was placed in the container such that it did not contact the PA solution directly. In other words, adsorption of PA by the paper took place in the gaseous phase.

Synthesis of Graphene Paper by Graphite Exfoliation

The graphene paper was obtained based on a procedure reported in K. Parvez et al., Journal of the American Chemical Society 2014, 136 (16), 6083-6091.

A graphite paper was used as a working electrode and platinum foils as counter electrode in a two-electrode system. 0.1 M of (NH₄)₂SO₄ solution (150 mL) was prepared as electrolyte for the graphite exfoliation. A DC voltage of 10V was applied to the electrodes for the electrochemical exfoliation of graphite paper. The suspended graphene was then collected by vacuum-assisted filtration on a cellulose filter membrane (pore size: 6 μm). The graphene sediment was then repeatedly washed by water and isopropyl alcohol. Later, the graphene sediment was dispersed in isopropyl alcohol by bath sonication for 4 hours. The well dispersed graphene suspension was centrifuged under 3000 rpm for 20 min. The supernatant was collected and stored in a reagent bottle under room temperature for further use. The graphene paper was prepared by the vacuum-assisted filtration of well-dispersed graphene on the PVDF filter membrane (pore size: 0.45μm).

Loading Amount and Changes Over Temperature

The adsorption of PA was directly measured by a gravimetric method. The loading amount of PA in the Ti₃C₂ MXene paper was calculated by the formula:

$$W \% = \frac{( W - {W}_{0} )}{{W}_{0}} \qquad ( 1 )$$

where W₀ refers to the original weight of Ti₃C₂ MXene paper and W refers to the weight after adsorption of PA.

The results show that the PA adsorbed under room temperature (25° C.) contributes around 5.95% of weight to the primitive Ti₃C₂ MXene paper. In one result, the weight of the primitive Ti₃C₂ MXene paper is 32.13 mg before adsorption, and 33.95 mg after adsorption of PA.

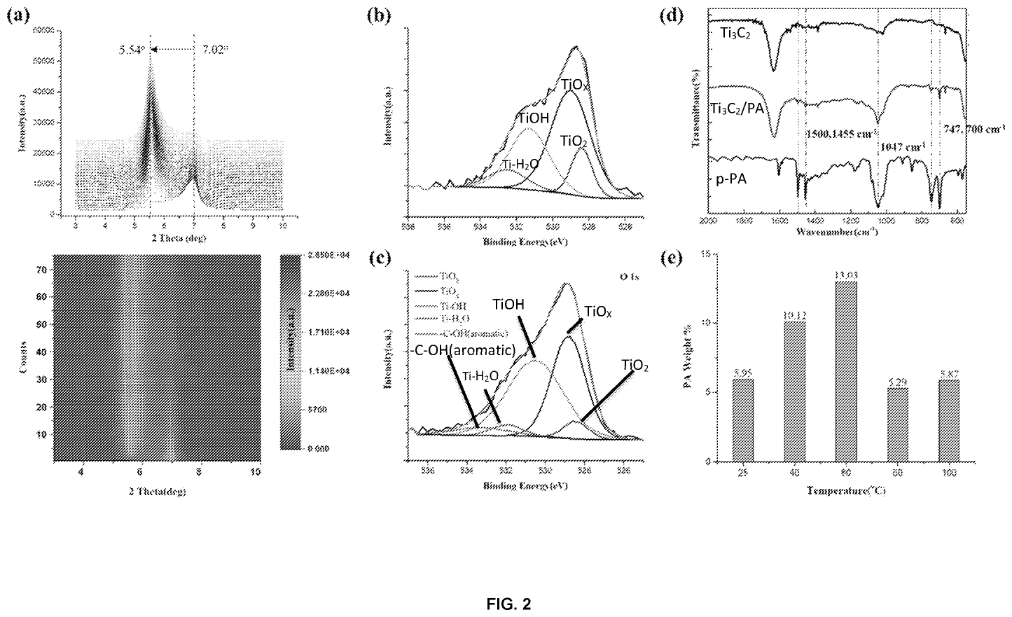

The adsorption of PA was also measured under increasing temperatures. PA adsorption increased with increasing temperatures and reaches the highest level at 60° C., which is about 13.03% of the original weight of MXene paper. However, as the temperature rose to 80° C. and 100° C., PA adsorption decreased significantly (FIG. 2e).

In-Situ XRD

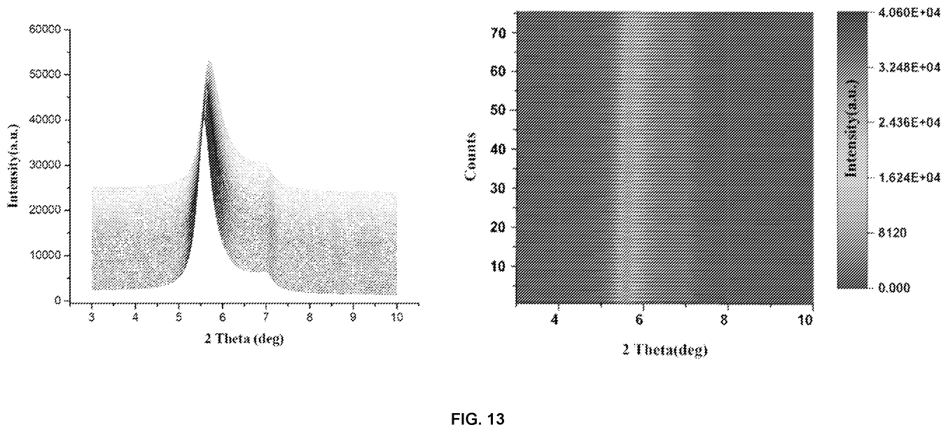

This technique was used to characterise the PA adsorption and the stability of Ti₃C₂/PA after a storage period of 5 hours in ambient condition (average humidity of 65% and 25° C.). FIG. 2a and FIG. 13 depict a selected range of XRD data compiled from stacking multiple scans over time. The detailed procedure is mentioned in Example 1.
The data confirmed a peak shift of (002) downward from 2θ=7.02° to 2θ=5.54, which suggests the expansion of d-spacing on Ti₃C₂ MXene for ≈3.25 Å.
It was observed that the peak shift occurred at the first 10 sweeps of scanning (corresponding to about cumulative 30 mins duration), which indicates favorable and rapid interaction between PA and the surface of Ti₃C₂ MXene.
After the adsorption of PA, there was a significant increase in XRD peak intensity. This phenomenon could be ascribed to the increased order of Ti₃C₂ MXene nanosheets along [001] by the intercalation of PA.
The presence of a weak original (002) peak in the XRD spectra could indicate residual nanosheets that were not intercalated with PA. According to Diao et al.'s work on slit pore deformation (R. Diao et al., The Journal of Physical Chemistry C 2016, 120 (51), 29272-29282), the expansion of interlayer space is thought to be induced by the positive solvation pressure before capillary condensation of PA molecules in the mesopore of Ti₃C₂ MXene paper, thus, leading to the deformation of interlayer space.

For comparison, graphene paper was subjected to the same PA adsorption process and characterised by XRD (FIG. 6). However, it was observed that there was no downward peak shift of (002), which indicates absence of storage of PA molecules in the graphene interlayer space.

The storage of PA in Ti₃C₂ MXene paper over a 4-month period was also monitored by XRD. As shown in the XRD pattern (FIG. 8a and FIG. 8b), the expanded d-spacing by the intercalation of PA molecules remained over a period of 4 months at ambient air. Although the d-spacing gradually decreased from 15.11 Å to 14.42 Å after 4 months, the layer distance was still 2 Å higher than the primitive Ti₃C₂ MXene paper. Also, the wider range XRD from 5° to 80° provides evidence that the PA/Ti₃C₂ MXene paper do not show significant oxidation over the period of 4 months (FIG. 8c).

Taken together, the Ti₃C₂ MXene displayed great potential for the long-term adsorption of molecules or VOCs.

XPS

This technique (PHI Quantera II) was applied to investigate the interaction between PA molecules and the surface of Ti₃C₂ MXene paper. It can detect the oxidation state of elements and chemical environment on the surface of materials. Core level analyses were targeted at C 1s, O 1s, Ti 2p, and F 1s, which were analyzed with the Gaussian/Lorentzian fitting curve using the CasaXPS software.

FIG. 2b and FIG. 2c show the XPS spectra of O 1s before and after PA adsorption in MXene paper. The O 1s spectra of primitive Ti₃C₂ MXene paper is composed of four components, TiO₂, TiOₓ, Ti—OH, Ti—H₂O, with their corresponding binding energy at 530.20 eV, 530.81 eV, 533.09 eV, and 534.30 eV, respectively. Notably, the binding energy of Ti—OH presents a significant red-shift up to 0.73 eV and the increase of the —OH content after the Ti₃C₂ MXene paper adsorbed the PA. This peak shift suggests the formation of hydrogen bonding between PA molecules and the —OH group on surface of the Ti₃C₂ MXene paper. A similar phenomenon was also reported in the system of interaction between SiO₂ and PVDF polymer (D. Yuan et al., Journal of Materials Chemistry C 2015, 3 (15), 3708-3713). The red-shift of the binding energy indicates a higher electron distribution around O atom of —OH group, meaning that the Ti₃C₂ MXene tends to act as hydrogen bond acceptor while interacting with the polar protic PA molecules.

FTIR

FTIR was also applied to verify the adsorption of PA in Ti₃C₂ MXene paper. The Ti₃C₂ MXene was ground with KBr powder and compressed as a small pellet for the FTIR measurement. The scan range was set from 400cm⁻¹⁻ to 4000cm⁻¹. The spectrum was accumulated with 8 times scanning. The resolution was set with 1 cm⁻¹ interval for data collection.

As shown in FIG. 2d, the spectrum of Ti₃C₂/PA shows new peaks at 700 cm⁻¹, 747 cm⁻¹, 1047 cm⁻¹, 1455 cm⁻¹ and 1500 cm⁻¹, which are ascribed to the benzene derivative peaks, C—H bending, C—O stretching, and C═C stretching, respectively. The result matches closely with the characteristic peaks in pure PA molecules and therefore confirms the adsorption of PA.

Contact Angle

Contact angles of Ti₃C₂ MXene paper was measured before and after adsorption of PA. The contact angle was measured by OCA 15 Pro. Typically, Ti₃C₂ MXene paper was fixed on a glass substrate. One drop of water was dropped on the surface of Ti₃C₂ MXene paper. The contact angle was then automatically generated by the software.

Contact angle of Ti₃C₂ MXene paper before PA adsorption is 31.6° . The contact angle of Ti₃C₂ MXene paper after PA adsorption is 65°,

### Example 3. Depositing Ti₃C₂ MXene on Silicon Substrate and its use for Detection of 1-Hexanol by Raman Spectroscopy

Silicon Substrate Procedure

A Si wafer was cleaned by acetone and isopropanol and dried with Argon gas. The dried Si wafer was then treated with O₂ plasma for 2 min with the RF power at 100 W. A suspension of Ti₃C₂ MXene (prepared from Example 1, 10 ml (1 mg/ml)) was provided in water (10 ml). This suspension was mixed with methanol at 1:1 volume ratio. The mixture of the Ti₃C₂ MXene suspension and methanol was added to chloroform at a 1:250 volume:volume ratio, and due to the immiscibility of the solvents, the nanosheets Ti₃C₂ MXene were reassembled at the interface between MXene/Methanol mixture and chloroform. The nanosheets were then collected from the interface of water and chloroform using a surface of the silicon substrate (Si wafer) and dried on a hot plate at 40 to 85° C. This provides the porous thin film layer of Ti₃C₂ MXene on silicon (Ti₃C₂ MXene/Si).

Experimental Procedure

The experimental procedure of Example 2 was repeated but with the use of 1-hexanol instead of PA.

Results

The resulting material after 1-hexanol adsorption was characterised by Raman spectroscopy. The Ti₃C₂ MXene deposited Si substrate was placed under Alpha300 SR confocal Raman spectroscopy. The 488 nm laser was selected for the characterization of the Ti₃C₂ MXene samples. The data accumulation time was set for 10 s and accumulated for 10 times. The grating number was set at 750 lines/mm. The magnification of objective lens was 20×. Clean Si substrate, Ti₃C₂ MXene/Si and pure 1-hexanol solvent were also characterised for comparison.

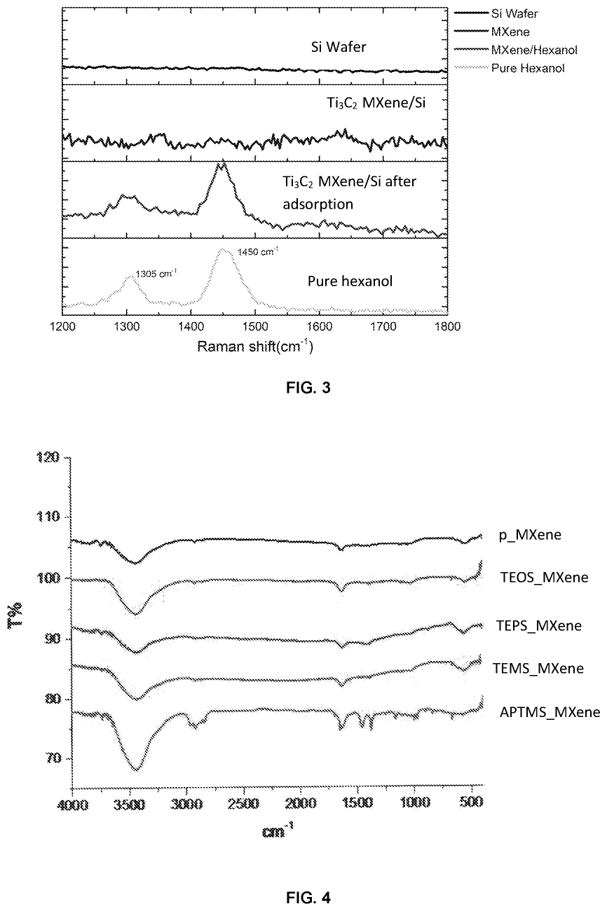

As shown in FIG. 3, the characteristic peak of 1-hexanol appeared on the Ti₃C₂ MXene/Si sample after 1-hexanol adsorption. The Ti₃C₂ MXene/Si sample did not show the peak of 1-hexanol before the adsorption process.

### Example 4. Surface Modification of Ti₃C₂ MXene

As the surface of the as-synthesised Ti₃C₂ MXene porous nanosheets contain various functional groups (—OH, —O—, —F), the nanosheets can be modified with hydrophobic compounds for enhanced or targeted binding with liquid or gaseous analytes.

In this example, the Ti₃C₂ MXene nanosheets was modified with various organosilanes, including 3-(aminopropyl)trimethoxysilane (APTMS), trimethoxy(octyl)silane (TEOS), trimethoxy(propyl)silane (TEPS) and trimethoxymethylsilane (TEMS). Functionalisation was confirmed using FTIR (FIG. 4).

Modification Procedure

The surface modification of Ti₃C₂ MXene was prepared by mixing 40 ml (1 mg/ml) of Ti₃C₂ MXene solution (as-obtained from Example 1) with 100 μl of 1% (v/v) organosilane ethanol solution. The reaction was conducted under room temperature with continuous stirring for 6 hr. The modified Ti₃C₂ MXene was subjected to vacuum filtration according to the procedure in Example 1 to obtain modified Ti₃C₂ MXene freestanding membrane except that the modified Ti₃C₂ MXene was filtered and washed by 100 ml of ethanol through the funnel. The modified Ti₃C₂ MXene was left on the funnel until the freestanding membrane dried out. The modified Ti₃C₂ MXene freestanding membrane was then peeled off from the filter membrane (the filter method is the same as those disclosed in Example 1). The surface modification method was repeated with each organosilane (APTMS, TEOS, TEPS, TEMS).

### Example 5. Detection of Plant VOCs with FTIR

Plants emit signaling chemicals in the form of VOCs. Therefore monitoring plant VOCs may allow crops to be protected from herbivores, pathogens and environmental stress, hence aiding in crop production. In the current example, alpha-pinene (α-Pinene), a type of terpene found in coniferous trees such as pine and aromatic dietary plants, was used as an analyte.

The experimental procedure of Example 2 was repeated except that PA was replaced with α-Pinene and a TEPS-modified Ti₃C₂ MXene paper (TEPS_MX-Pin; prepared from Example 4) was used.

After gaseous adsorption of α-Pinene in a sealed container, the paper was characterised by FTIR (procedure is same as Example 2).

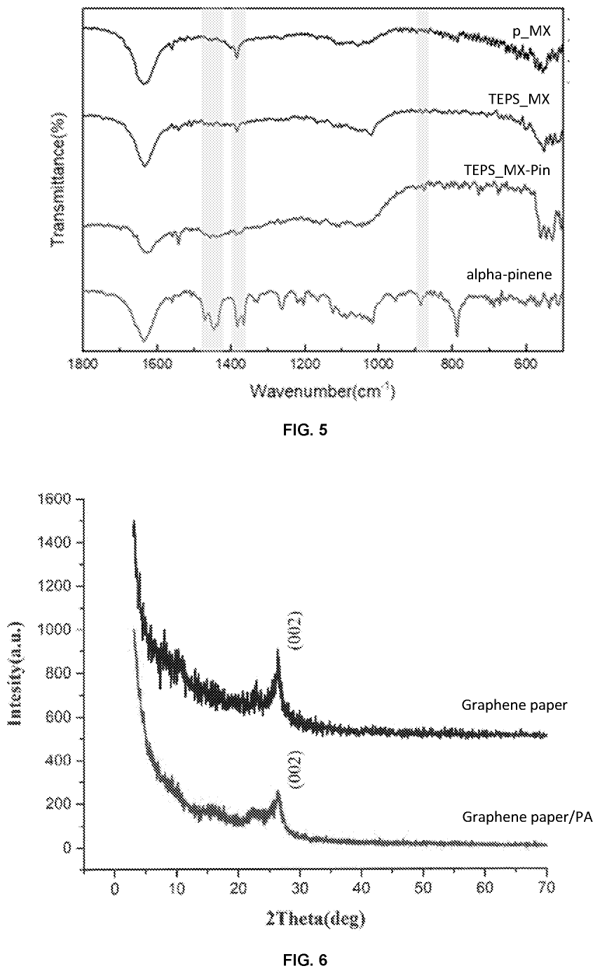

As shown in FIG. 5, there are new peaks at highlighted area, 1465 cm⁻¹, 1450 cm⁻¹, 1385 cm⁻¹ to 1380 cm⁻¹ and 895 cm⁻¹ to 885 cm⁻¹, corresponding to the —CH₂ bending, —CH₃ bending, —CH bending of gem dimethyl group and C═C bending. The new characteristic peaks match closely with those of pure α-Pinene and therefore confirmed the adsorption α-Pinene using modified MXene.

### Example 6. Preparation of Ti3C2 MXene Heater and its Thermal Stability

The thermal stability of the Ti₃C₂ MXene paper was investigated and characterised in this example. Ti₃C₂ MXene has intrinsic electron conductivity and therefore can be effectively heated when a voltage is applied to it.

Preparation of Ti₃C₂ MXene Heater

Ti₃C₂ MXene paper (1.5 cm×1.5 cm; prepared in accordance with Example 1) was connected by a copper tape at opposing ends. Silver paste (25 μL at each end/electrode) was used to improve the connection in between the copper tape and Ti₃C₂ MXene paper. The copper tape acted as electrodes for the resistive heating experiment.

Thermal Stability

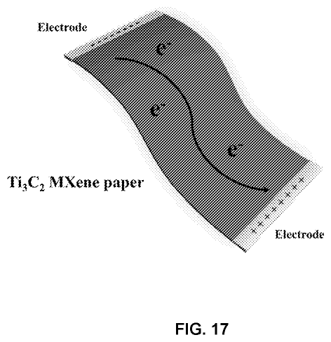

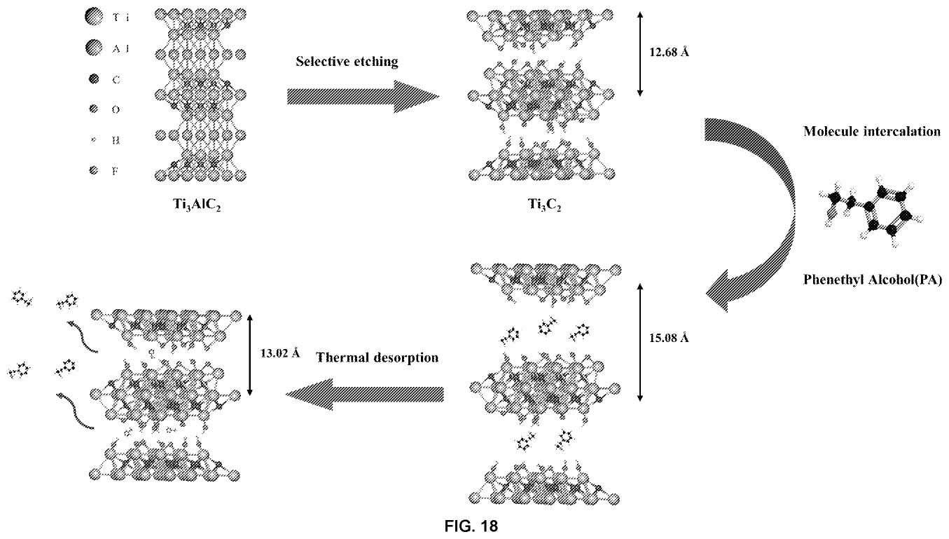

Temperature stability measurements were performed by mounting both copper electrodes of Ti₃C₂ MXene paper on a glass slide or suspending the freestanding Ti₃C₂ MXene paper outwards in accordance with the schematic shown in FIG. 17. Voltage was provided by connecting both terminal electrodes to a DC supplier. Different voltages were then applied for monitoring the temperature stability as a function of time. The temperature was recorded by a IR detector (Fluke).

As shown in FIG. 9a, the Ti₃C₂ MXene paper can be heated up to 35° C., 60° C., 100° C. and 130° C. when applying 1.0 V, 1.5 V, 2.0 V and 2.5 V respectively. The thermal stability was also monitored by the IR camera. As shown in FIG. 9b, the increase in temperature to the designated temperature occurs within 1 second upon applying each voltage state.

When a higher voltage was applied to the Ti₃C₂ MXene paper, there was a larger fluctuation of temperature at roughly ±10° C. The temperature fluctuation at lower voltage level consistently shows lower interference at roughly ±3° C. In addition, the Ti₃C₂ MXene paper can withstand at least 20 minutes of continuous heating, showing the promising thermal stability.

Compared with other film heaters, the Ti₃C₂ MXene heater requires only 0.3 s and 1.5 V to attain a temperature of 50° C. (Table 3). It is also operational when in a flexible state.

TABLE 3
Comparison of film heaters. Each reference number in square
brackets refers to the corresponding citation below.

| | | | | Heating | | |
|---|---|---|---|---|---|---|
| | | | | Rate to | Sheet | |
| | | Thickness | | 50° C. | resistance | |
| Materials | Transparent | (μm) | Flexible | (sec)/V | (Ω/sq) | Ref. |
| Ti₃C₂ MXene | No | 12.78 | Yes | 0.3/1.5 | 0.61 | — |
| Ag nanowire | Yes | 100 | Yes | 10/5 | 25 | ^{[1]} |
| polymer | | | | | | |
| composite | | | | | | |
| Ag nanowire | Yes | 50 | Yes | 10/6 | 5.60 | ^{[2]} |
| polyimide | | | | | | |
| composite | | | | | | |
| Ag nanoparticle | Yes | 15 | Yes | 7/15 | 3.47E−3 | ^{[3]} |
| ink/PET | | | | | | |
| Graphene/PET | Yes | 188 | Yes | 5/12 | 43 | ^{[4]} |
| ITO/PET | Yes | 130 | Yes | 250/20 to | 392 | ^{[4]} |
| | | | | 47° C. | | |
| SWCNT/PET | Yes | 188 | N/A | 3/6 | 356 | ^{[5]} |
| MWCNT/epoxy | No | 700 | No | 10/40 | 1428.57 | ^{[6]} |
| composite | | | | | | |
| CuNi/PES | Yes | 200 | Yes | 3/3 | 16.2 | ^{[7]} |

^{[1]} J. Li et al., Macromolecular Materials and Engineering 2014, 299 (11), 1403-1409.
^{[2]} Q. Huang et al., RSC Advances 2015, 5 (57), 45836-45842.
^{[3]} J. S. Park et al., Nanotechnology 2018, 29 (25), 255302.
^{[4]} J. Kang et al., Nano letters 2011, 11 (12), 5154-5158.
^{[5]} Y. H. Yoon et al., Advanced Materials 2007, 19 (23), 4284-4287.
^{[6]} B.-K. Zhu et al., Composites Science and Technology 2006, 66 (3-4), 548-554.
^{[7]} H.-J. Kim et al., Journal of Materials Chemistry A 2015, 3 (32), 16621-16626.

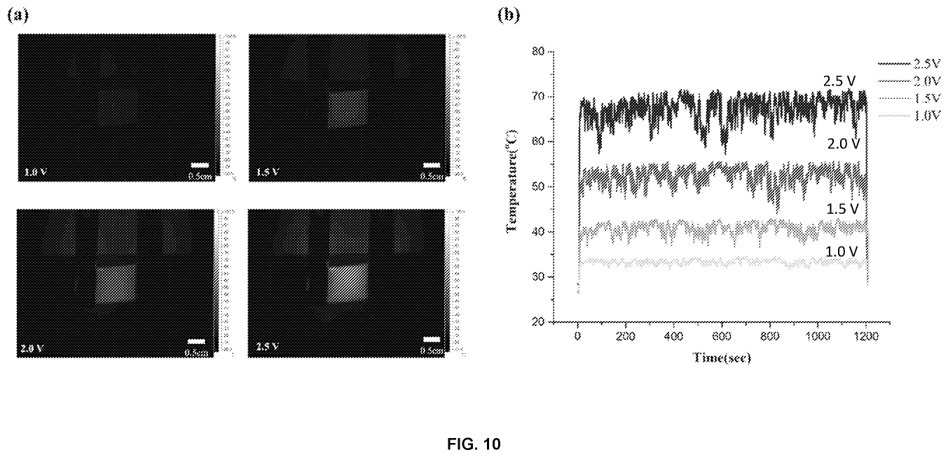

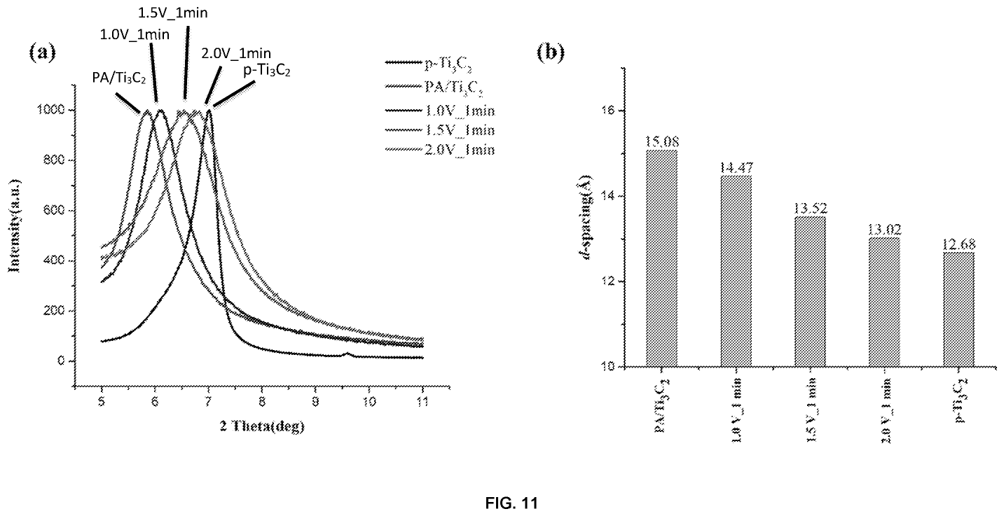

As a further comparison, graphene paper (made according to procedure in Example 2 also shows a fast response time on resistive heating. However as shown in FIG. 10, the highest temperature attained was 70° C. when 2.5 V was applied, and there were more temperature fluctuations compared with Ti₃C₂ MXene paper.

### Example 7. Use of Ti₃C₂ MXene Paper for Release of PA

The release of PA from Ti₃C₂ MXene paper was monitored by XRD and Headspace Gas Chromatography-Mass Spectrometry (HS-GCMS). PA was adsorbed by a Ti₃C₂ MXene paper in accordance with the procedure in Example 2, and the PA/Ti₃C₂ MXene paper was then attached with copper tape based on the procedure mentioned in Example 6 and heated by applying different voltages to release PA.

XRD

The XRD spectra shows the obvious up-shift of basal peak as higher voltages were applied (FIG. 11a). Based on Bragg's law, the d-spacing was calculated from the 2θ of the PA/Ti₃C₂ MXene. There was a decrease of d-spacing from 15.08 Å to 13.02 Å when the voltage increased from 1.0 V to 2.0 V (FIG. 11b). The collapse of the d-spacing in Ti₃C₂ MXene was attributed to the thermal desorption of the PA molecules.

However, it was noted that use of higher voltages results in the oxidation of the Ti₃C₂ MXene. After the Ti₃C₂ MXene was heated to 2.5 V at roughly 130° C., the characteristic peak of anatase phase TiO₂ appears at around 2θ=25°. Thus, the Ti₃C₂ MXene heater was operated at voltages below 2.0 V.

HS-GCMS

The residual PA from Ti₃C₂ MXene paper was released and quantified using HS-GCMS. Before the HS-GCMS measurement, the Ti₃C₂ paper was resistively heated with different level of voltages to release the PA, after which the residual PA was measured by the HS-GCMS. By subtracting the released amount of PA from original PA/Ti₃C₂ MXene paper from the results from each level of heating, the average release of PA can be calculated.

Agilent 7697A/5977A (Agilent Technologies, USA) was used to conduct the quantitative analysis of the PA release. The DB-5MS column (length: 30 m, diameter: 0.25 mm, film thickness: 0.25 μm) was used to separate the chemical compounds. The oven temperature was programmed and started from 40° C. with the rate 10° C. min⁻¹ to 230° C.; in addition, the equilibrium time was holding for 2 minutes at the initial and terminal temperature. The transfer line temperature between GC and MS system was at 250° C. The equilibrium temperature for PA extraction at headspace sampler was set at 120° C. for 5 minutes. The carrier gas, helium gas, was controlled at flow rate of 1.2 ml min⁻¹. The selective range of m/z was setting from 30 to 450. The quantitative analysis was performed by Masshunter (Agilent Technologies, USA).

Tables 4 and 5 show the linear range, linearity, detection limit (LOD), quantitative limit (LOQ), precision and result of HS-GCMS analysis. The calibration curve can be linearly fitted with R²=0.994 at the range of 500 to 8000 ppm. In addition, based on the function of the calibration curve, LOD and LOQ were determined to be 182.03 ppm and 551.61 ppm, respectively, which are lower than the level of the analytes. The precision of the calibration curve at the standard of 2000 ppm was at 7.88%. The release of residual PA using PA/Ti₃C₂ paper, 1.0 V and 1.5 V are 1423.38±254.47 ppm, 284.41±74.32 ppm and 101.39±6.52 ppm, respectively. The result for paper applied with 2.0 V is not reliable due to the signal to noise ratio far below 3. The relative release of PA in percentage was calculated to be ≈80% at 1.0 V, ≈92% at 1.5 V, and ≈100% at 2.0 V. The HS-GCMS results indicated that the majority of PA molecules were released to the atmosphere once a low voltage was applied.

TABLE 4
The linearity of calibration curve and
sensitivity of the headspace GCMS analysis.

| Validation Parameters | Phenethyl Alcohol |
|---|---|
| Linear Range (ppm) | 500-8000 |
| R² | 0.9994 |
| Calibration Curve | y = 203.10x-32006.47 |
| LOD^{a} (ppm) | 182.03 |
| LOQ^{b} (Ppm) | 551.61 |
| % RSD of three duplicates | 7.88 |
| standard at 2000 ppm | |

^{a}LOD = 3.3*S_{b}/slope of the function, S_{b} = Standard Deviation of Residual
^{b}LOQ = 10*S_{b}/slope of the function, S_{b} = Standard Deviation of Residual

TABLE 4
The results of PA release from PA/Ti₃C₂
paper at different voltages (n = 3).

| | Release |
|---|---|
| | amount |
| | of residual |
| PA/Ti₃C₂ paper | PA (ppm) |
| 1.0 V | 284.41 ± 74.32 |
| 1.5 V | 101.39 ± 6.25 |
| 2.0 V | N/A^{a} |
| Pristine PA/Ti₃C₂ | 1423.38 ± 254.47 |

^{a}Result not available: S/N < 3

### Example 8. Application of Ti₃C₂ MXene on Other Substrates

As shown by the SEM images in FIG. 12, the hydrophilic nature of Ti₃C₂ MXene allows superb affinity to hydrophilic substrates such as O₂ plasma treated Si wafer (FIG. 12a) or gold nanoparticles (FIG. 12b). The method for depositing the MXene on the substrates is the same as those described in Example 3.

1. A material suitable for the adsorption, storage and release of volatile organic compounds comprising:
a porous thin film layer formed from nanosheets of one or more MXenes.

2. A volatile organic compound storage device, comprising:
a material suitable for the adsorption, storage and release of volatile organic compounds comprising a porous thin film layer formed from nanosheets of one or more MXenes; and
electrodes attached to a surface of the porous thin film layer formed from nanosheets of one or more MXenes.

3. The volatile organic compound storage device according to claim 2, wherein one or both of the following apply:
(aa) the electrodes are provided in the form of a metal tape on a surface of the porous thin film layer formed from nanosheets of one or more MXenes; and
(bb) the volatile organic compound storage device is configured to release a volatile organic compound stored in said device by way of resistive heating.

4. The material according to claim 1 wherein the material is provided solely as a free-standing porous thin film layer.

5. The material according to claim 1 wherein the material further comprises a substrate material having a surface and the porous thin film layer is formed on the surface of the substrate material.

6. (canceled)

7. The material according to claim 1, wherein the one or more MXenes have a minimum lateral size of 0.22 μm.

8. The material according to claim 7, wherein the one or more MXenes have a minimum lateral size of 0.45 μm.

9. The material according to claim 1, wherein each nanosheet has a thickness of from 1 nm to 6 nm, m.

10. (canceled)

11. (canceled)

12. (canceled)

13. The material according to claim 1, wherein the one or more MXenes is selected from one or more of the group consisting of Ti₂C, (Ti_{0.5}, Nb_{0.5})₂C, V₂C, Nb₂C, Mo₂C, Mo₂N, (Ti_{0.5}, Nb_{0.5})₂C, Ti₂N, W_{1.33}C, Nb_{1.33}C, Mo_{1.33}C, Mo_{1.33}Y_{0.67}C, Ti₃C₂, Ti₃CN, Zr₃C₂, Hf₃C₂, Ti₄N_{3,} Nb₄C₃, Ta₄C₃, V₄C₃, Mo₄VC₄, Mo₂TiC₂, Cr₂TiC₂, Mo₂ScC₂, and Mo₂Ti₂C₃.

14. (canceled)

15. (canceled)

16. (canceled)

17. (canceled)

18. A method of forming a free-standing porous thin film layer of an MXene as described in claim 1, comprising the steps of:
(a) providing a suspension of porous nanosheets of an MXene in a solvent;
(b) subjecting the suspension of porous nanosheets of an MXene to vacuum filtration with a filter membrane to provide a porous thin-film layer on a surface of the filter membrane; and
(c) removing the porous thin-film layer from the surface of the filter membrane to provide the free-standing porous thin film layer of an MXene.

19. (canceled)

20. A method of forming a porous thin film layer of an MXene on a surface of a substrate material, the method comprising the steps of:
(A) providing a suspension of porous nanosheets of an MXene in a first solvent;
(B) adding the suspension to a second solvent, where the first and second solvents are immiscible, to provide the porous nanosheets of an MXene at an interface between the first and second solvents;
(C) collecting the porous nanosheets of an MXene from the interface of the first and second solvents using a surface of a substrate material to which the porous nanosheets of an MXene are attachable to, to provide the porous thin film layer of an MXene on a surface of a substrate material.

21. (canceled)

22. (canceled)

23. A method of forming a volatile organic compound storage device as described in claim 2, comprising the step of forming two or more electrodes on a surface of:
(AA) a freestanding porous thin film layer formed from nanosheets of one or more MXene; or
(AB) a porous thin film layer formed from nanosheets of one or more MXene on a substrate.

24. (canceled)

25. A method of detecting an analyte comprising the steps of:
(BA) exposing a material suitable for the adsorption, storage and release of volatile organic compounds as described in claim 1 to an environment where the analyte is suspected to be present for a period of time; and
(BB) subsequently subjecting the material suitable for the adsorption, storage and release of volatile organic compounds to spectroscopic and/or gravimetric analysis to determine the presence or absence of the analyte in said environment.

26. (canceled)

27. (canceled)

28. (canceled)

29. An olfactory display system comprising at least one volatile organic compound storage device as described in claim 2.

30. The volatile organic compound storage device according to claim 2, wherein the material is provided solely as a free-standing porous thin film layer.

31. The volatile organic compound storage device according to claim 2, wherein the material further comprises a substrate material having a surface and the porous thin film layer is formed on the surface of the substrate material.

32. The volatile organic compound storage device according to claim 2, wherein the one or more MXenes have a minimum lateral size of 0.22 μm.

33. The volatile organic compound storage device according to claim 32, wherein the one or more MXenes have a minimum lateral size of 0.45 μm.

34. The volatile organic compound storage device according to claim 2, wherein each nanosheet has a thickness of from 1 nm to 6 nm.

35. The volatile organic compound storage device according to claim 2, wherein the one or more MXenes is selected from one or more of the group consisting of Ti₂C, (Ti_{0.5}, Nb_{0.5})₂C, V₂C, Nb₂C, Mo₂C, Mo₂N, (Ti_{0.5}, Nb_{0.5})₂C, Ti₂N, W_{1.33}C, Nb_{1.33}C, Mo_{1.33}C, Mo_{1.33}Y_{0.67}C, Ti₃C₂, Ti₃CN, Zr₃C₂, Hf₃C₂, Ti₄N₃, Nb₄C₃, Ta₄C₃, V₄C₃, Mo₄VC₄, Mo₂TiC₂, Cr₂TiC₂, Mo₂ScC₂, and Mo₂Ti₂C₃.
